(19) United States
(12) Patent Application Publication
(10) Pub. No.: US 2026/0305745 A1
(43) Pub. Date: Oct. 8, 2026

(54) SYSTEM, METHOD, AND APPARATUS FOR BIOINSPIRED STRATEGIES FOR EFFICIENT LIGHT HARVESTING AND OPTICAL PROCESSING

(71) Applicant: Trustees of Tufts College, MEDFORD, MA (US)

(72) Inventors: Fiorenzo G. Omenetto, Lexington, MA (US); Giulia Guidetti, Somerville, MA (US)

(21) Appl. No.: 19/545,962

(22) Filed: Feb. 20, 2026

Related U.S. Application Data
(63) Continuation of application No. PCT/US2024/043616, filed on Aug. 23, 2024.
(60) Provisional application No. 63/578,325, filed on Aug. 23, 2023.

(51) Int. Cl. A41G 1/00; C07K 14/435

(52) U.S. Cl. CPC A41G 1/00; C07K 14/43586

(57) ABSTRACT

Producing a replica of a leaf includes casting a silk fibroin solution directly on the leaf, allowing the silk fibroin solution to dry at room temperature to form a silk film, and after the silk film is dry, peeling the silk film from the jewel orchid leaf to obtain the replica. The leaf may have a lattice structure including cells having a partially-spherical portion.

### CLAIM TO PRIORITY

This application claims benefit of and is a continuation of International Patent Application No. PCT/US2024/043616 (Attorney Docket No. 2095.0626), filed Aug. 23, 2024, and entitled “SYSTEM, METHOD, AND APPARATUS FOR BIOINSPIRED STRATEGIES FOR EFFICIENT LIGHT HARVESTING AND OPTICAL PROCESSING,” International Pub. No. WO 2025/043173, which is hereby incorporated by reference in its entirety for all purposes.

International Patent Application No. PCT/US2024/043616 relates to, incorporates by reference for all purposes, and claims priority to U.S. Provisional Application No. 63/578,325 (Attorney Docket No. 2095.0540), filed Aug. 23, 2023.

### STATEMENT REGARDING FEDERALLY SPONSORED RESEARCH

This invention was made with government support under N00014-19-1-2399 awarded by the Department of Defense. The government has certain rights in the invention.

### BACKGROUND

Plants and animals produce highly evolved, hierarchical systems that combine physicochemical material parameters with functional micro- and nanostructures to manage energy, motion, and species survival. These systems, besides providing cues on bottom-up manufacturing of technological structures, can give insight on strategies for efficient energy management.

Effective light management is crucial for the survival of living systems, aiding essential functions like heat regulation and energy harvesting. Exploring biological systems that flourish in extreme environments reveals untapped opportunities for novel, optimized radiation regulation strategies. As frequently reported in literature, these systems often exhibit structural colors—colors derived not from pigments but from micro- and nanoscale structures that interact with light—to achieve advanced optical management. Overall, the regulation of solar radiation in these systems typically involves intricate photochemical processes intertwined with hierarchical biomaterial structures. Together, these elements enable efficient harvesting, modulation, filtering, and redistribution of light.

In plants, the outer structure of the epidermis serves diverse functions, including light harvesting regulation, attracting pollinators, and promoting seed dispersal. In leaves, epidermal cells can regulate light intake, thus strongly affecting the overall photosynthetic behavior of the plant. These cells typically exhibit irregular shapes and sizes and limited information is available regarding the morphological adjustments of these cells in response to varying light conditions in their habitat. Reports in the literature describe that for some plants thriving in low-light environments the outer epidermal cells have evolved conical shapes, potentially acting as focusing elements to enhance light concentration onto chloroplasts, thus leading to increased photosynthetic efficiency. The role of cell morphology and cell arrangement on light harvesting, that could influence the plant's photosynthetic activity and, ultimately, its growth, remains largely unexplored.

### SUMMARY

Plants living in low light conditions challenge traditional light harvesting norms: instead of vertically focusing light onto chloroplast, they redistribute it laterally through an optically interconnected network of round cell. This strategy enhances photon redistribution, revealing a specialized approach for thriving in dim environments, as demonstrated by bio-inspired biomaterial-based replicas with potential applications in light redistribution.

An example method of producing a replica of a leaf having a lattice structure including cells having a partially-spherical portion includes casting a silk fibroin solution directly on the leaf, allowing the silk fibroin solution to dry at room temperature to form a silk film, after the silk film is dry, peeling the silk film from the jewel orchid leaf to obtain the replica.

An example method of producing a positive replica of a leaf having a lattice structure including cells having a partially spherical portion may include casting an elastomeric material solution (e.g., a polydimethylsiloxane (PDMS) solution) directly on the leaf; allowing the PDMS solution to dry at room temperature to form a mold; after the mold is dry, peeling the mold from the leaf.

An example replica of a leaf having a lattice structure including cells having a partially spherical portion may include a biopolymer matrix having a matrix lattice structure corresponding to the lattice structure of the leaf, wherein the biopolymer matrix includes at least one optical property corresponding to an optical property of the leaf.

An example method of manufacturing the replica is disclosed.

In the disclosed methods, the leaf is living and survives the method. In the disclosed methods, the lattice structure includes cells having a hemispherical portion.

These and other systems, methods, objects, features, and advantages of the present disclosure will be apparent to those skilled in the art from the following detailed description of the preferred embodiment and the drawings.

References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1. Optical and morphological characterization of Macodes petola orchid leaves—(A) Macroscopic picture of a jewel orchid Macodes petola leaf showing a golden reticulated vein pattern alternating with dark green regions. (B) Low magnification bright field reflection micrograph of a portion of the leaf displaying round epidermal cells. (C) High magnification bright field reflection micrograph of a green region displaying closely packed cells. (D) Cryo-scanning electron microscopy (Cryo-SEM) image of a cross-section portion of the leaf showing round cells in the upper epidermis. False color green, blue, and orange overlays highlight upper epidermal cells, palisade cells, and the vascular bundle, respectively. Inset shows a schematic representation of the cells morphology and packing in the upper epidermis. (E) Cryo-SEM image capturing the leaf section's curvilinear structure (top, side view) and the round morphology of the epidermal cells (bottom, side view). (F) Top-view cryo-SEM image depicting the tight packing of the cells and an example of the local 6-fold arrangement (false green color overlay).

FIG. 2. Collective light propagation in Macodes leaves—(A) Size distribution of the Macodes epidermal cells radius. Reported values are mean±standard deviation (SD) for N cells. (B) Bright field reflection micrograph of a representative region of Macodes leaf (top) and corresponding centroids position (bottom) used to compute the leaf lattice. (C) Radial distribution function computed for Macodes short-range order array. Arrows indicate peaks representative of short-range order. (D) Angular distribution function for Macodes short-range order array with 6-fold lobe pattern characteristic of hexagonal packing. (E) Bright field reflection micrograph of Macodes leaves in top view, showing the propagation of light through networked cells for the ideal case of a reference cell surrounded by 6 other cells. (F) Corresponding leaf lattice showing the position of the centroids associated to each cell. The reference cell (ref) is in the center, surrounded by 6 nearest-neighbour (nn) cells, next nearest-neighbour (nnn) cells, and next next nearest-neighbour (nnnn) cells. (G) Schematic representation of the retroreflected lights from the top of each cell (blue circles) and of the light planarly exchanged through cross-communication between adjacent cells (yellow circles; yellow dashed lines are drawn as visual aid). (H) Schematic representation of networked cell communication between reference (ref) and nearest neighbors (nn) refractive elements (cells). When a single cell is illuminated with a narrow beam of light focused on top of the cell and normal to it (ray 0 on the ref cell), the light is retroreflected normally to the cell (blue ray); conversely, if the incoming light hits the cell on the side (ray 1), specularly reflected light can be horizontally redirected to a neighboring cell before being reflected vertically away from it (yellow ray). (I) Effect of numerical aperture in illumination on the light propagation pattern observed among the cells. An increase in the numerical aperture in illumination results in a larger portion of the cells being illuminated and in a larger angular spread of the incoming light, leading to larger illumination spots (blue circles) and laterally redistributed reflection spots (yellow lines). Side-view and top-view schematics of in-plane lateral light redistribution among cells (left panels), and corresponding bright-field micrographs; 3D schematic representation of the collective behavior of light redistribution on the leaf. (J) Ray tracing simulation of light interaction with an assembly of spherical particles. For large illumination cones, the light reflected off the ref particle strongly bends horizontally and is distributed to the nearest neighbor particles (nn).

FIG. 3. Biopolymer-based optical network replica—(A) Comparison of light redistribution between a silk positive replica of Macodes leaf (top row) and an unpatterned smooth silk film (bottom row). Bright field reflection micrographs showing cross-communication spots characteristic of light redistribution across the microdomes for the silk replica and lack of thereof in the smooth silk film (OM, top-view). Corresponding cross-section (SEM, side-view) and top-view (SEM, top-view) SEM images of the films. Macroscopic pictures of laser light spreading as collected on a white screen (laser, top-view). Schematic representation of the setup used to investigate the light redistribution with laser light (λ=650 nm) with relative orientation of the silk films with respect to the laser (top row, black arrow indicates laser propagation direction) and corresponding ray-tracing simulations for red light. (B) Distribution plots of the laser power attenuation after the interaction with the silk films reported as transmitted power. Each distribution plot reports the mean±standard deviation for N=3 measurements. (C) Far-field diffraction pattern (left) and corresponding pattern with highlighted conventional diffraction points for an ideal hexagonal lattice (right, white dots). Diffraction spots corresponding to the 0^{th}, 1^{st}, 2^{nd} and 3^{rd} order are indicated. Schematic representation of the setup used to investigate the symmetry of the replica lattice: the replica was illuminated with laser light (λ=633 nm) and the far-field diffraction pattern was collected on a white screen.

FIG. 4. Optical response of the leaves of plants from the Orchidaceae genus: Macodes petola, Ludisia discolor, Anoectochilus roxburghii, and Phalenopsis phantom (common orchid). From left to right, macroscopic appearance of a portion of the leaf, bright field reflection image with numerical aperture (NA) in illumination fully closed; bright field reflection image with numerical aperture (NA) in illumination fully open; dark field reflection image; bright field cross-section. For the cross-section adaxial (ad) and abaxial (ab) sides are indicated; insets show schematic representation of the various shapes of the upper epidermis outer cells for the leaves of each investigated species.

FIG. 5. Size distribution of the epidermal cell radius. Reported values are mean standard deviation (SD) calculated from N measurements. (A) size distribution calculated for the leaves of 5 plants globally, while (B-F) report the size distribution for each of the selected plants individually.

FIG. 6. Evaluation of the lattice order in an ideal hexagonal lattice and in 3 Macodes petola plants (plant 1, plant 2, and plant 3). (A) Code-generated hexagonal lattice and bright field reflection micrographs of the leaves of 3 Macodes petola plants showing representative regions with short-range order cells arrangement. (B) Corresponding lattice organization for the hexagonal lattice (code-generated) and for Macodes petola plants. Black dots correspond to the centroid position for each cell of the plant. (C) Corresponding radial distribution function (RDF) calculated from the position of the centroids of each lattice. Black arrows indicate significant peak positions indicative of short-range order. (D) Corresponding angular distribution function calculated from the position of the centroids of each lattice.

FIG. 7. (A, D) Bright field reflection image of two regions of Macodes petola leaves showing a tightly packed short-range order hexagonal lattice with in-plane light redistribution between the cells through cross-communication (blue and yellow rectangles). (B, C, E, F) Details of cells showing 6 fundamental cross-communication reflection spots with neighboring cells (white ellipses), caused by the short-range hexagonal packing. Additional spots due to variation of the cells' morphology and packing are also visible (blue squares).

FIG. 8. Light path analysis on Macodes leaf. (A) Selected leaf region showing cells arranged in a 6-fold symmetry pattern; (B) corresponding overlayed light paths (blue, yellow, and purple) and cells outline (grey). (C) Leaf lattice computed from the centroid position of each cell; highlighted are a representative reference cell (ref), the nearest-neighbor cells (nn), and the next nearest-neighbor cells (nnn) for that cell. (D) Leaf lattice with highlighted the retroreflected light (blue spots). (E) Leaf lattice with highlighted the light path for cells that are nearest neighbors (yellow circles indicate positions on the cells where light is reflected, yellow dashed lines indicate light path). (F) Leaf lattice with highlighted the light path for cells that are next nearest neighbors (purple circles indicate positions on the cells where light is reflected, purple dashed lines indicate light path).

FIG. 9. Cross-communication spots as a function of numerical aperture of the light source for an ideal hexagonal lattice (first column), a region of Macodes leaf (second column), and for the silk positive replica (third column). Macodes and replicas images were acquired in bright field reflection with either low (first row) or high (second row) numerical aperture (NA) in illumination.

FIG. 10. (A) Normalized fluorescence emission spectra as a function of wavelength for silk-rhodamine 6G film positive replica and silk-rhodamine 6G film (smooth unpatterned film); excitation wavelength λ=480 nm, bandwidth=40 nm. (B) Corresponding top-view optical RGB images (top row), top-view black-and-white images extracted for λ=544 nm (middle row) from multispectral cubes, and cross-section scanning electron images (bottom row). (C) Bright field reflection of silk-rhodamine 6G film positive replica showing cross-communication spots due to the transferred micropattern.

FIG. 11. (A) Far-field diffraction pattern of a M. petola silk replica (left), and corresponding pattern with overlayed conventional diffraction pattern points (right, white dots) for the 6-fold symmetry (right, dashed lines). Diffraction spots corresponding to the 0^{th} up to the 7^{th} order are visible and indicated. (B) Gray value intensity as a function of distance along profile A in panel (A).

FIG. 12. A further example of optical characterization of Macodes petola orchid leaves. (A) Macroscopic picture of the jewel orchid Macodes petola. (B) Detail of a leaf showing a shiny golden reticulated vein pattern. (C) Low magnification bright field reflection micrograph of a portion of the leaf showing round golden cells (chlorophyll (chl) depleted cells) and round green cells (chlorophyll-rich cells). (D) High magnification bright field transmission micrograph of a cross-section portion of the leaf showing round cells in the upper epidermis. (E) High magnification top-view micrograph showing chl-depleted and chl-rich cells. (F) Normalized reflectance with respect to a silver mirror as a function of wavelength of the chl-depleted cells, showing broadband reflectance (plotted curve is average±standard deviation, N=20). (G) Multispectral analysis: low magnification false-color recombined picture of the Macodes petola plant and corresponding reflectance spectra of the chl-depleted and chl-rich cells, normalized with respect to a white reference. High magnification RGB image in bright field reflection of a portion of a leaf showing chl-depleted and chl-rich domains and corresponding false-color images of the composite (recombined), the chl-rich (green), and chl-depleted (yellow) cells. (H) Top view cryo-SEM image of a portion of the cells showing the round morphology and tight packing of the cells. Inset shows cross-section high magnification cryo-SEM image of a single cell. (I) Macroscopic picture of a leaf under UV light (λ=365 nm) showing red fluorescence in correspondence of the chl-rich regions and lack of it in correspondence of the veins (left). Details of the lack of fluorescent from the veins (right).

FIG. 13. A further example of collective light propagation in Macodes leaves. (A, B) Bright field reflection micrographs of a portion of Macodes leaves in top view showing light propagation through networked cells. Focus is either (A) at the base of the cells or (B) on top of the cells. (C) Schematic representation of the networked cell communication between nearest neighbors (nn) cells. When a single cell is illuminated with a narrow beam of light focused on top of the cell and normal to it (ray 0 on the central cell and on the nearest neighbor cell nn), the light is specularly reflected back normally to the cell (orange ray); conversely, if the incoming light hits the cell on the side (ray 1 on the nearest neighbor cell nn), the specularly reflected light can be horizontally redirected to a neighboring cell before being reflected vertically away from it (yellow ray). (D) Effect of numerical aperture (NA) in illumination on the light propagation pattern observed among the cells. Both for chl-depleted and chl-rich cells an increase in the NA in illumination results in a larger portion of the cells being illuminated and thus in larger reflection spots of laterally redistributed light. (E) Wavelength-dependent laser light propagation (left) and combined white light illumination and laser illumination (right) across the leaf Photosynthetically relevant radiation (λ=632 nm) is selectively absorbed by pigmented cells and propagates along the plane of the leaf through unpigmented cells (top row). Radiation not photosynthetically relevant (λ=543 nm) propagates across the leaf radially from the incident point. (F) Schematic representation of the hexagonal lattice forming the optical network in Macodes leaves. Unpigmented cells are represented in yellow, while pigmented cells in green. The light distribution is radiation dependent and directed by the arrangement of pigmented cells across the leaf. (G) Schematic representation of the setup used for FDTD modelling and corresponding electromagnetic fields interaction with a hexagonal array of cells illuminated by white light, and light with central wavelength λ=632 nm and λ=543 nm.

FIG. 14. A further example of biopolymer-based optical network replica. (A) Fabrication process of the silk replica of the Macodes leaf top surface pattern. (B) Bright field reflection micrograph of the replica showing the light redistribution across the cells. (C) SEM image in top view (left) and in cross-section (right) of the replica. (D) Schematic representation of the setup used to investigate the symmetry of the replica lattice: the replica was illuminated with laser light (λ=632 nm) and the far-field diffraction pattern was collected on a white screen. (E) Far-field diffraction pattern (left) with highlighted conventional diffraction pattern points for the 6-fold symmetry (right). (F) Transmission images of laser light (λ=650 nm) propagating through silk films. Schematic representation of the setup with relative orientation of the silk films with respect to the laser (top row) and corresponding transmission images captured on a white screen (bottom row). (G) Distribution plots of the laser power attenuation after the interaction with the silk films reported as transmission power. Each distribution plot reports the mean±standard deviation.

FIG. 15—#1—how living optical networks work in a variety of plants that grow in low-light conditions. #2—use of optical models to predict in-plane light redistribution and assessment of corresponding photosynthesis efficiency; Study of plant and functional adaptation by growing plants under various light conditions. #3—fabrication and optimization of plant-inspired, biologically based replicas, their characterization and testing, and development of conformal, flexible, photovoltaic.

FIG. 16. (A) Traditional understanding of vertical focusing of the light in epidermal cells favors light concentration on the chloroplasts. (B) Schematic representation of light coupling in photonic microdroplets and corresponding micrograph. (C) Observed in-plane cross-communication mechanism in liquid crystal microparticles as a function of illumination conditions.

FIG. 17. Macodes petola orchid show leaves with round epidermal cells arranged in a short-range order hexagonal lattice that promotes cross-communication and in-plane light redistribution.

### DETAILED DESCRIPTION

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “silk fibroin” refers to silk fibroin protein whether produced by silkworm, spider, or other insect, or otherwise generated (Lucas et al., Adv. Protein Chem., 13: 107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

Disclosed herein is an optically interconnected network formed by the cells of the leaves of the Macodes petola jewel orchid that provides light harvesting and redistribution across its surface. The planar redistribution of light incident on the leaf is enabled by the distinctively rounded shape of the outer epidermal cells and by their arrangement in a short-range ordered hexagonal lattice. Successful replication of a live leaf in a free-standing biopolymer film validates the observation and opens avenues for the exploration of non-traditional energy harvesting interfaces that are soft, conformal, and curvilinear.

Disclosed herein is a unique cellular arrangement found in the leaves of the jewel orchid species Macodes petola. The cells have a favorable combination of shape, size, and are connected through an ordered structure that makes them able to distribute the light across the leaf surface through a “living optical network”. The structure is reminiscent of previously reported optical networks formed by ordered planar assemblies of microparticles in liquid crystal and photonic systems, as of yet undocumented for natural systems.

Morphological and optical measurements, analysis, and simulations suggest a structure-function relationship that optimizes energy redistribution in the form of a soft, flexible, curvilinear format, efficiently harvesting and redirecting light within its leaves. Protein-based replicas of the leaf structure confirm the findings while providing a platform to explore bioinspired, conformal, flexible solar harvesters and photoconversion systems.

An example method according to an embodiment of the present disclosure may produce a replica of a leaf. In some examples, the leaf may have a lattice structure including cells having a partially-spherical portion. In some examples, the leaf may be from a jewel orchid plant.

The example method may include casting a silk fibroin solution on (e.g., directly on) the leaf, and allowing the silk fibroin solution to dry (e.g., at room temperature) to form a silk film. In some examples, the silk fibroin solution may have a concentration of between 2 wt % and 20 wt % or between 2 wt % and 15 wt %. For example, the concentration may be C=8.48 wt %. For concentrations that are very low (e.g., under 2 wt %), the silk film may be too thin and may not correctly replicate the morphology of the leaf, while for higher concentrations (e.g., above 15 wt % or above 20 wt %), the silk solution may be viscous, and the fidelity of the reproduced details of the leaf may be too low. After the silk film is dry, the silk film may be peeled from the leaf to obtain the replica.

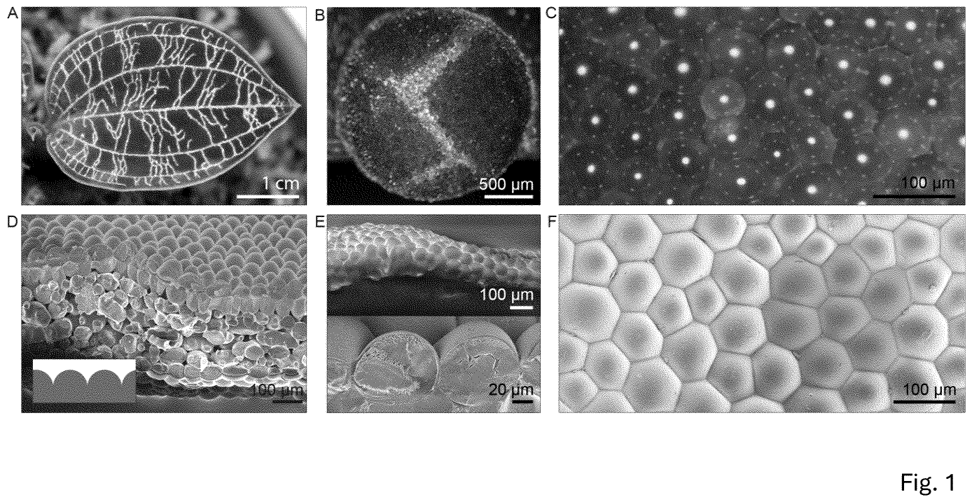

In some examples, the leaf may be from a jewel orchid species from a genus selected from the group consisting of genus Anoectochilus, Dossinia, Goodyera, Ludisia, Macodes, Rhomboda, and Odontochilus. Leaves from a jewel orchid may be very shiny with a metallic appearance, as shown by examples in FIG. 1A.

In some examples, the leaf may be from the Macodes petola jewel orchid. The Macodes petola leaf may have an optical response unique to jewel orchids that may be transferred from the leaf to the silk film upon drying of the silk film. The leaf from the Macodes petola jewel orchid may have outer epidermal cells with a semi-spherical shape (e.g., a round morphology) that are arranged in a quasi-hexagonal lattice. However, embodiments are not limited thereto, and as described herein, other leaves may be used in the example method. Leaves used according to example methods may have outer epidermal cells with a conical shape that are randomly distributed over an x-y plane of the leaf.

The replica according to the example method may have a replica lattice structure corresponding to the lattice structure of the leaf. In some examples, light propagation through the replica may result in a far field diffraction pattern associated with a regularity of the replica lattice structure of the replica. In some examples—for example, when the lattice structure of the leaf is hexagonal or quasi-hexagonal, such as it may be for the Macodes petola jewel orchid—the far field diffraction pattern may include a central peak surrounded by six other maxima.

In some examples, a symmetry of the far field diffraction pattern may depend on a pattern of the leaf. A leaf may not be damaged by the silk solution and may remain alive and healthy during and after the example method. Indeed, example embodiments may be used to replicate micro to nano features of living systems such as leaves without affecting them.

The example method may include tuning a parameter of the lattice structure of the replica to control light guiding and redistribution. For example, tuning may be optionally performed by tailoring one or more properties or components of the silk fibroin solution or adjusting a degree of crystallinity of the replica. As an example, the replica's mechanical properties may be tuned by tuning a composition of the silk solution, such as by adding a plasticizer such as glycerol, or the composition of the silk solution may be tuned, such as by pigment addition, silk concentration, or silk molecular weight. A replica crystallinity may be tuned by water vapor annealing of the silk film once it is peeled off the leaf.

The example method may include tuning a cell shape of the replica lattice structure of the replica to control light guiding and redistribution.

In example embodiments, the lattice structure of the replica may include a hexagonal pattern and a round morphology corresponding to the leaf. For example, the shape of the outer cells of the Macodes petola leaf may be very round, and (roughly) hemispherical, as shown in FIG. 1F. The hemispherical shape of the Macodes petola cells—rather than the conical morphology of other jewel orchid leaves—combined with their arrangement in a quasi-ordered lattice, may be what grants the in-plane light redistribution in the leaf, and therefore, in the replica.

In some examples, the replica may be a negative replica of the leaf.

In some examples, the replica may be a free-standing film with an integrated optical network.

The example method may include obtaining the silk fibroin solution by sieving silk fibroin powder through a sieve (e.g., a 300 μm mesh size sieve), dissolving the sieved silk fibroin powder in double distilled water to produce an intermediate solution, centrifuging the intermediate solution (e.g., at 10,200 rpm for 20 minutes at 4° C.) to produce a clear supernatant, and collecting and filtering the clear supernatant to obtain the silk solution.

The example method may include obtaining the silk fibroin solution by dissolving and solubilizing silk cocoons.

The example method may include obtaining the silk fibroin solution by dissolving a silk powder in water.

An example method according to an embodiment of the present disclosure may produce a positive replica of a leaf. In some examples, the leaf may have a lattice structure and/or cells as described herein. In some examples, the leaf may be from a jewel orchid plant. The example method may include casting an elastomeric material solution (e.g., a polydimethylsiloxane (PDMS) solution) on (e.g., directly on) the leaf, allowing the elastomeric material solution (e.g., the PDMS solution) to dry (e.g., at room temperature) to form a mold, and after the mold is dry, peeling the mold from the leaf.

In some examples, the leaf may be from a jewel orchid species from a genus selected from the group consisting of genus Anoectochilus, Dossinia, Goodyera, Ludisia, Macodes, Rhomboda, and Odontochilus. In some examples, the leaf may be from the Macodes petola jewel orchid.

The mold may have a mold lattice structure corresponding to the lattice structure of the leaf.

The example method may include casting a silk fibroin solution on the mold, allowing the silk fibroin solution to dry (e.g., at room temperature) to form a silk film, and after the silk film is dry, removing the silk film from the mold to obtain the positive replica.

The example method may include tuning a parameter of the replica lattice structure of the positive replica to control light guiding and redistribution. For example, a parameter of the replica lattice structure may be tuned as described for the lattice structure according to other example embodiments herein.

The example method may include tuning a cell shape of the replica lattice structure of the positive replica to control light guiding and redistribution.

The example method may include tuning a composition of the silk fibroin solution to control light guiding and redistribution.

The positive replica may be a free-standing film with an integrated optical network.

The silk fibroin solution may have a concentration of between 2 wt % and 15 wt %. For example, the concentration may be C=8.48 wt %. For concentrations that are very low (e.g., under 2 wt %), the silk film may be too thin and may not correctly replicate the morphology of the leaf, while for higher concentrations (e.g., above 15 wt %), the silk solution may be viscous, and the fidelity of the reproduced details of the leaf may be too low.

The example method may include obtaining the silk fibroin solution by sieving silk fibroin powder through a sieve (e.g., a 300 μm sieve), dissolving the sieved silk fibroin powder in double distilled water to produce an intermediate solution, centrifuging the intermediate solution (e.g., at 10,200 rpm for 20 minutes at 4° C.) to produce a clear supernatant, and collecting and filtering the clear supernatant to obtain the silk solution.

The positive replica may have a lattice structure corresponding to the lattice structure of the leaf.

The lattice structure of the positive replica may include a hexagonal pattern and a round morphology corresponding to the leaf. For example, the shape of the outer cells of the Macodes petola leaf may be very round, and (roughly) hemispherical, as shown in FIG. 1F. The hemispherical (rather than conical) shape of the Macodes petola cells, combined with their arrangement in a quasi-ordered lattice, may be what grants the in-plane light redistribution in the leaf, and therefore, in the positive replica.

Light propagation through the positive replica may result in a far field diffraction pattern associated with a regularity of the lattice structure of the positive replica. The far field diffraction pattern may include a central peak surrounded by six other maxima.

The example methods described herein may include obtaining the silk fibroin solution by dissolving and solubilizing silk cocoons.

The example methods described herein may include obtaining the silk fibroin solution by dissolving a silk powder in water.

A replica of a leaf (e.g., a leaf having a lattice structure) according to an example embodiment of the present disclosure may include a biopolymer matrix having a matrix lattice structure corresponding to the lattice structure of the leaf. The biopolymer matrix may include at least one optical property corresponding to an optical property of the leaf. The replica may be formed by one or more of the example methods described herein.

The biopolymer matrix may include a silk protein.

The replica may be a free-standing film with an integrated optical network.

A parameter of the matrix lattice structure of the biopolymer matrix may be tuned to control light guiding and redistribution. For example, a parameter of the matrix lattice structure may be tuned as described for the lattice structure according to example embodiments herein.

A cell shape of the matrix lattice structure of the biopolymer matrix may be tuned to control light guiding and redistribution.

A composition of the biopolymer matrix may be tuned to control guiding and redistribution.

The leaf may be from a jewel orchid species from a genus selected from the group consisting of genus Anoectochilus, Dossinia, Goodyera, Ludisia, Macodes, Rhomboda, and Odontochilus. In some examples, the leaf may be from the Macodes petola jewel orchid.

In some examples, the replica may be a positive replica of the leaf. In some examples, the replica may be a negative replica of the leaf.

The matrix lattice structure of the biopolymer matrix may include a hexagonal pattern and a round morphology corresponding to the leaf. For example, the shape of the outer cells of the Macodes petola leaf may be very round, and (roughly) hemispherical, as shown in FIG. 1F. The hemispherical (rather than conical) shape of the cells, combined with their arrangement in a short-range order lattice, may be what grants the in-plane light redistribution in the leaf, and therefore, in the replica.

Light propagation through the replica may result in a far field diffraction pattern associated with a regularity of the lattice structure of the replica. The far field diffraction pattern may include a central peak surrounded by six other maxima.

The at least one optical property of the biopolymer matrix may include at least one of an optical network corresponding to an optical network of the leaf, a metallic appearance corresponding to a metallic appearance of the leaf, or a retroreflection corresponding to a retroreflection of the leaf.

The at least one optical property may include an in-plane redistribution of light incident on the biopolymer matrix guided by the lattice structure of the biopolymer matrix.

The replica may be structured to be used for light management. The light management may include at least one of: an in-plane omnidirectional light coupler, an optical switch that receives a single input and routes the single input to different sets of regularly spaced outputs, a wavelength-selective optical communication switch that enables wavelength-dependent light propagation along pre-determined paths, a wavelength-selective optical network, an optical security tag, a retroreflector that redirects incident beams into laterally reflected beams with a regular pattern, or an optical network system having a reconfigurable optical property and a controlled degradation.

The light management may be at least one of the wavelength-selective optical network, the omnidirectional optical coupler, or the optical security tag.

The replica may be structured to be a flexible coating for handling light through the at least one optical property.

The replica may include an absorber in the biopolymer matrix to direct light propagation within an optical network of the biopolymer matrix. The absorber may include a dopant that absorbs light in a predetermined wavelength range.

The replica may include a coating on the biopolymer matrix to induce total internal reflection and increase an efficiency of light transmission in an optical network of the biopolymer matrix.

The biopolymer matrix may include spatially-dependent variations in optical transparency to have patterned diffusive/scattering regions.

An example optical system according to an embodiment of the present disclosure may include a replica as described with reference to embodiments herein. The replica may be optically coupled to a light source, an optical waveguide, an optical detector, or a combination thereof. For example, the optical system may include a replica optically coupled to a wavelength-selective optical waveguide in which, by shining visible light onto the replica, some wavelengths (e.g., red) propagate further in the x-y directions compared to others (e.g., green).

An example method according to an embodiment of the present disclosure may manufacture a replica as described with reference to one or more embodiments herein.

An example method according to an embodiment of the present disclosure may manufacture an optical system as described with reference to one or more embodiments herein.

In an example method described with reference to one or more embodiments herein, the leaf may be living and survive the method.

In an example method described with reference to one or more embodiments herein, the lattice structure may include cells having a hemispherical portion.

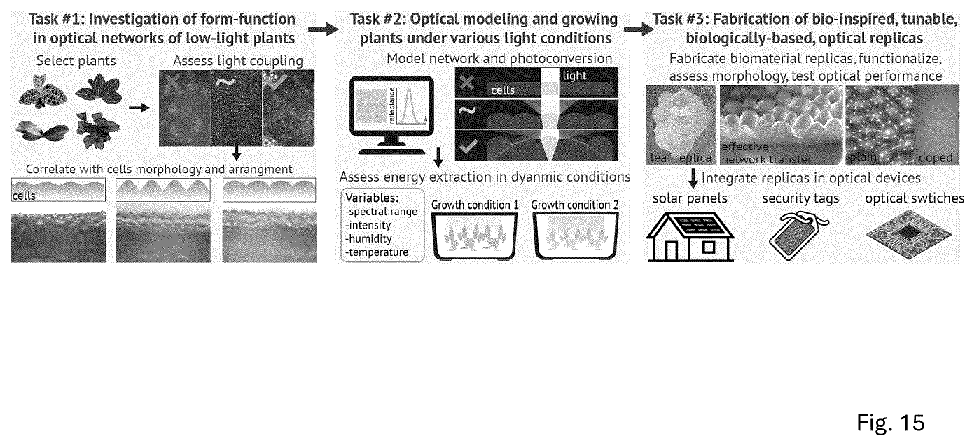

Disclosed herein are the structure-function relationships governing energy harvesting and redistribution strategies used by plants living in low light conditions to inspire new paradigms for biomaterial-based, flexible, conformal, smart photoconverting interfaces. Disclosed herein is the discovery of a novel light harvesting mechanism present in plants that seem to have evolved specific micro-to-nanostructures functionally dedicated to efficient energy harvesting in adaptation to the challenging low-light environments in which they grow. This disclosure is organized in terms of 3 sections (FIG. 15). Section #1 investigated and characterized plants living in low-light conditions by describing the newly observed phenomenon in which light propagates on the leaf plane instead of through the leaf, demonstrating an increase in photon dwelling time on the leaf and, thus, a corresponding increase in photosynthesis rate. Section #2 explored the variation of the plants' growing conditions to induce plant adaptation and fine-tuning of the light harvesting and redistribution mechanism in their leaves; theoretical models were developed to support the experimentally observed light coupling in the leaves. Section #3 translated the research findings into prototypes in the form of curvilinear, soft, conformal film replicas based on biomaterials (regenerated silk fibroin solutions). These replicas can be used for a variety of light harvesting and photoconversion strategies based on photon collection and redistribution in systems such as solar cells, security tags, and optical switches. Disclosed herein is how plants use singular cellular patterns in their leaves to collect enough solar energy that enables them to thrive in challenging environments.

This disclosure concerns light harvesting and redistribution mechanisms in natural systems, using plants that grow in low-light conditions (jewel orchids) as model organisms. The newly observed light management phenomenon on which this disclosure is based on is the following: leaves of certain plants have round cells organized in a short-range lattice that forms an optically interconnected network. Each cell is observed to exchange light with neighboring cells, contributing to a collective light redistribution system. Briefly, each illuminated cell focuses a portion of the light on the photosynthetic centers present within each cell; another portion of the light is reflected laterally to other cells on the leaf so that it can be further absorbed by other chloroplasts not directly illuminated. By doing so, a higher portion of the light is collected by the chloroplasts in the leaves compared to the amount of light that would be harvested if the cells did not display this cooperative behavior (no lateral redistribution, only focusing through the leaf). The disclosure herein includes: (1) demonstrating a mechanism of light harvesting in plants that diverges from the commonly accepted description of structure-function for light management, (2) advancing the field of plant science by establishing how and why certain biological systems growing in low light have evolved a distinctive patterned epidermis, and (3) introducing a new class of soft, conformal materials with a plant-like cellular network that can be used for efficient solar energy solutions.

More specifically, novel insights on photosynthesis studies include how plants adapt and thrive in extreme environmental conditions by having cells with round morphologies closely-packed in an ordered array. Dome-like cells are expected to enable a longer photon dwelling time within each cell of the leaf (photons resonate within each cell) and to promote photons distribution from one cell to its nearest cell neighbor (following geometrical optics). The in-cell resonance is expected to increase the amount of light absorbed by chloroplasts in one single cell, while the lateral redistribution across different cells promotes photons absorption by chloroplasts not directly illuminated by the light. Managing radiation is, indeed, crucial for living systems to regulate heat and to harvest energy. The investigation of the structure-function relation of the cellular arrangement in leaves also improves the understanding of how other species (both plant and animal) survive in low-light environments and can reveal any symbiotic relationships. Plant-pollinator research themes also benefit from this disclosure, as the optical appearance of some plant organs is linked to interspecies communication and species survival.

From an optical standpoint, this disclosure concerns a theoretical framework to describe light-coupling in microscale natural networks, a concept presently lacking in scientific literature. This fundamental disclosure provides the scientific foundation for the design of plant-inspired biomaterials that replicate the form-factors associated with light harvesting of certain plants. This disclosure has significant technological potential that can be used to imagine new strategies for light management, based on natural structures that collect and redistribute photons for increased photosynthetic efficiency by increasing photon-photosynthetic centers interaction (higher photon dwelling time) through the structure itself. New enabled possibilities include flexible, conformal, photovoltaic panels that can be directly integrated within soft surfaces like garments or skin, omnidirectional light couplers and switches, wavelength selective optical networks, and cryptographic systems for all-optical processing or security applications such as physically unclonable security tags. These forms will be assembled using regenerated silk fibroin as a sustainable, bioactive material platform. Regenerated silk is an ideal material given its demonstrated capacity to be reshaped into micropatterned, flexible, free standing films that can be easily doped with biologically active molecules, such as natural photosynthetic compounds, providing a platform for further investigation of novel light-harvesting and photoconversion approaches. The impact of this disclosure is multifold since it challenges and advances the plant science field, by redefining light harvestings across diverse species; it can be translated from the academic to the industrial world, thus becoming a concrete step towards clean energy harvesting solutions. The disclosure supports the long-term goal of fostering and promoting scientific interest in the world that surrounds us, increasing our understanding of natural systems while contributing to the development of sustainable and innovative materials for planetary stewardship.

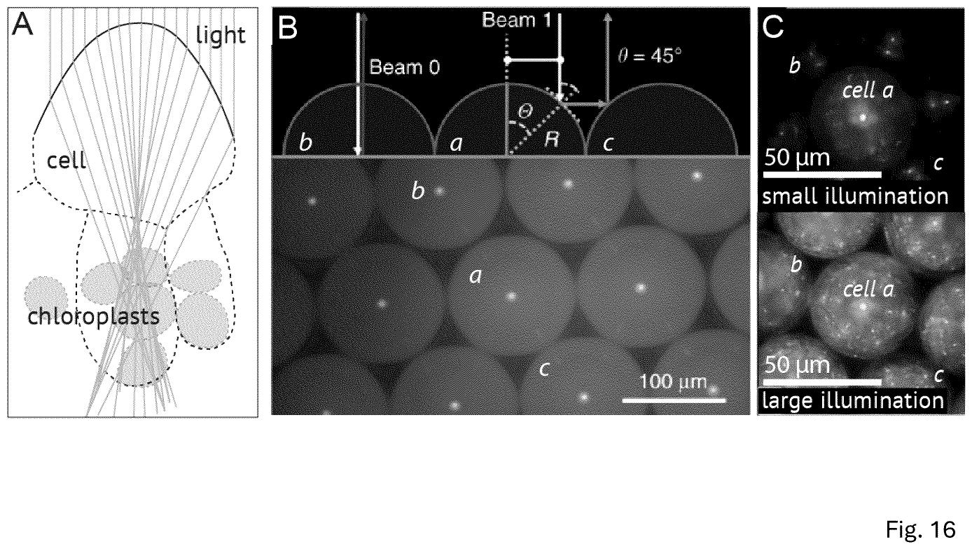

Traditional understanding of plant light-harvesting strategies suggests that plants use the outer cells in their leaves (epidermal cells) to focus light in the vertical direction onto the photosynthetic centers (the chloroplasts) laying just underneath, in a localized manner, without any documented lateral light redistribution (FIG. 16A). This implies that only the chloroplasts that are directly illuminated can capture light and contribute to photosynthesis. The leaves of certain plants growing in low-light conditions have epidermal cells with a conical morphology (rather than a flat or a convex morphology), aiding in further focusing light vertically onto chloroplasts, as observed for the petals of some flowers. This indicates that the amount of light that reaches the chloroplasts largely depends on how much light strikes on a single cell and on the morphology of that cell, with no known relationship with the size and the microscale packing of those cells, and how that affects the photoconversion efficiency. There are no reports so far of plants that have leaves that exhibit simultaneously (i) regularly-sized round-cells and (ii) their arrangement in a short-range lattice, thus missing the requirements needed for the formation of a so-called “living optical network”. In terms of conspicuous optical effects on leaves, plants can modulate the morphology of their epidermis to produce metallic reflections or unusual blue colors, yet the description of the physical structures responsible for such phenomena remains limited. Despite the abundance of tropical low-light plants with metallic leaves, the connection between this unusual optical appearance and its biological and energetic relevance has barely been explored.

The regular planar assembly of spherical micro-particles of the same size can result in the lateral redistribution of light from one microparticle to neighboring ones, thus forming a so-called optical network (FIG. 16B-16C); the larger the angular distribution of the incoming light and the scale at which the microparticles are assembled in a regular pattern, the more intense and extended is the laterally-redistributed light; this induces a stronger cross-communication effect of light bouncing across the cells of the network. Network systems formed by just a few tens of particles have been observed in bottlebrush block-copolymer and raspberry-like microparticle systems, and, most notably, in chiral nematic liquid crystal assemblies. Their characterization has been, so far, limited to either reporting the cross-communication phenomenon, or it has focused mostly on the role of the chiral assembly of the liquid crystals forming each particle (type of liquid crystal, reflected wavelength, effect of the refractive index of the surrounding phase). Optically, what is largely missing is a theoretical framework providing a comprehensive description of the fundamental geometric optics and network behavior that induces light coupling in microscale networks (effect of size of the particles, polydispersity, type and order degree of the lattice). There is also little exploration of how light propagates inside the elements forming the network and how that affects the interaction with any photoreactive material present in each element.

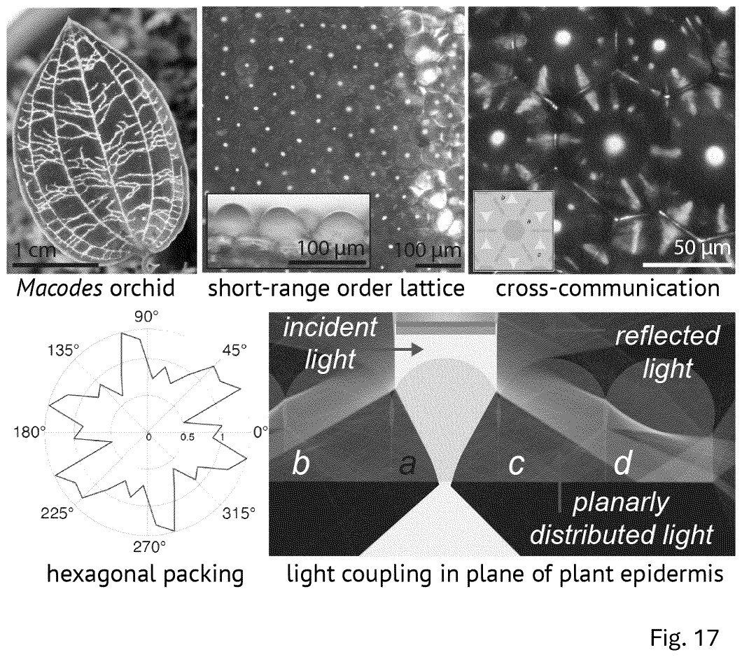

Jewel orchids are a family of orchids with distinctive foliage appearance, characterized by several metallic features and net-like patterns; they include genera such as Macodes, Ludisia, and Anoectochilus. These plants are typically found in environments characterized by high humidity, with varying light conditions with predominantly low and diffused light and, more rarely, intense light in the form of sunflecks. The disclosure herein challenges the concept of solely vertical light focusing through the leaves, revealing that certain jewel orchids living in low light conditions, present on their leaves a convergence of three key factors namely (1) round-shaped cells, with (2) low polydispersity, and arranged in (3) a short-range order hexagonal lattice. This forms an optically interconnected cellular network that promotes horizontal light redistribution on the plane of the leaf (FIG. 17). The observed cell arrangement aligns with mechanisms previously documented for self-assembled optical networks made of ordered lattices of spherical microdroplets, yet this similarity has not been characterized or documented. Statistical investigation on one jewel orchid species (Macodes petola) indicated the presence of a network of cells arranged in an ordered pattern forming a short-range order lattice.

This unique combination of cell morphology, size, and arrangement in a networked structure in the M. petola species enables effective light harvesting and redistribution in the horizontal direction, across the leaf surface. When one of such leaves is illuminated with visible light, a discernible short-range order pattern, characterized by bright reflection spots, emerges across the cell network on each cell's sides. This pattern exhibits 6-fold symmetry, indicating the formation of an optically coupled hexagonal network within the leaf. By doing so, a significant increase in photosynthetic efficiency is expected to occur, given the ability to propagate photons to leaf cells not directly illuminated and to further trap them horizontally within the individual round cells of the network. Due to this arrangement, cells efficiently redistribute light laterally between adjacent domains in the plane of the leaf. The cooperative optical coupling mechanism observed in this living optical network suggests an adaptation to dark environments, facilitating optimal light delivery to chloroplasts through cells' cross-communication along with geometry-induced increased photon dwelling time in those photosynthetic centers. Notably, this phenomenon of cross-communication between cells of a leaf has not been described in natural systems before, potentially due to the lack of simultaneous coexistence of the needed cell shape, size, and packing.

This disclosure bridges plant science and optics, employing optical methods and engineering rigor to investigate the behavior of regular patterns found in plants with disruptive consequences for both fields and a new fundamental understanding of photon management in plants that may be used to direct the fabrication of plant-inspired biomaterial-based counterparts for light harvesting and redistribution.

Formation, Hierarchy, and Function of Optical Network Structures in Plants that Grow in Low-Light Environments.

Discovery and understanding of the formation, multiscale arrangement, and biological function of the optical network structures in plants that grow in low-light conditions allows the fundamental exploration of a so-far unreported mechanism of light harvesting and photoconversion in plants, possibly linked to their thriving in such environments. This disclosure lays the foundation for optically co-opting plant growth mechanisms and enabling large-scale growth.

Methods—(1) Select low-light plants (and corresponding regular-light and high-light plants to be used as controls) based on preliminary studies on jewel orchids and established literature on leaves' optical appearance of various genera of plants, considering the illumination conditions of their natural environment. Orchids genera that are promising to display optically interconnected cellular networks include Macodes, Ludisia, and Anoectochilus (low-light), with Brassavola, Vanda, and Spathoglottis genera used as controls (medium- to high-light). Non-orchid genera with metallic appearance indicative of light coupling include Begonia, Pilea, Tradescantia, Alocasia, and Anthurium. For each selected genera identify at least 2 species (as applicable) and for each species acquire at least 3 plants to have a statistically significant number of leaves available per species (15+) for initial investigation. This selection of genera provides enough species to screen for cell morphology and arrangement that are most likely to show light coupling effect. (2) Cultivate and propagate plants in situ through tissue culturing techniques in custom-built cabinets with controlled relative humidity (RH), temperature (T), and illumination conditions. This step allows co-opting the growth mechanism of the optically responsive structures found in the plants and consistently and rapidly allows obtaining large amounts of plants all with the same genetic material to ensure the highest uniformity in the optical properties. Plants are first tissue cultured using commercial kits to generate enough plantlets to carry out experiments (5/species); multiplication and rooting media are used to maximize yield of plantlets (expected 80% plantlet formation for each propagated tissue within 2 months). To carry out experiments, plantlets need to be removed from the sterile environment of the culture and transferred in soil. Once plantlets are established to soil (~1 month), grow at illumination, RH, and T conditions comparable to their original thriving environments: for tropical plants RH~70-95% and T ~26° C. while for non-tropical plants RH~40-70%, T~20° C. Low-light plants are illuminated with full-spectrum LED lights with 40-80 photosynthetic photon flux density (PPFD), medium-to-high lights plants with LEDs with 80-350 PPFD. The so grown plants serve for optical and morphological investigation of their leaves and for the fabrication of biopolymer-based replicas. (3) Investigate light propagation in leaves using optical microscopy, spectroscopy, and supercontinuum laser imaging, examining both single cell and collective network responses. In vivo optical microscopy (using stereo and custom-made microscopes that can accommodate living plants) determines cells' shape, size, and arrangement on the leaves screening for the presence of any short-range or long-range order that can give rise to a lattice (which is crucial for the light coupling in the network). Specifically, bright-field reflection (BFR) microscopy at low (2×, 5×) magnification assesses the cells' arrangement at large scale (over m²-mm²); at high magnification (20×, 50×) BFR microscopy studies light coupling patterns among few cells (size, reflectance, number, and arrangement of the cross-communication lines/spots) and morphology and size of each cell. In vivo optical spectroscopy quantifies the leaves' reflectance properties and the interplay between reflected, scattered, absorbed, and transmitted light; stand-alone spectrometers with optical fibers and multispectral cameras are used to quantify the amount of light reflected along the plane of the leaf in individual region of interests (single cells) and over large areas (multiple cells), respectively. Expected planarly reflected light varies ~40-70%, with ~15% absorbed, ~5% transmitted and ~5% scattered for plants with cell arrangement behaving as optical networks. Plants with no optical network reflect less light within the plane of the leaf (~15-30%) with correspondingly higher scattered and transmitted light. Quantification of these parameters provides an optical assessment of the efficiency of the networks. In vivo supercontinuum laser imaging investigates the presence of any wavelength-dependent pattern on the leaves (light of only a specific wavelength propagating along a specific direction) by illuminating them with laser light with various wavelengths both within (e.g., λ_{blue}~480 nm, λ_{red}~633 nm) and outside the photosynthetic relevant ranges (e.g., λ_{green}~532 nm). These patterns, when present, derive from heterogeneous distribution of pigments in the leaf plane (chlorophyll λ_{absorption}~450 nm and λ_{absorption}~675 nm, anthocyanin λ_{absorption}~270 nm and λ_{absorption}~520 nm). These measurements determine the most promising plant species whose leaves cells' morphology and pattern induce light coupling. (4) In depth morphological and chemical analysis of the plants with demonstrated optical networks and/or any further relevant optical response. To do so, use cryo-scanning-electron microscopy (cryo-SEM) to study thin leaves sections (microtome to a thickness ~2-5 μm) in cross-section (for single cell morphology and cell arrangement in the thickness of the leaves) and punched leaf portions in top-view (for cells' packing in the plane of the leaf) and cross-check with optical microscopy results. Cryo-SEM with in situ Raman spectroscopy and elemental analysis through Energy Dispersive X-ray (EDX) spectroscopy is used to determine the multiscale arrangement and the composition of the leaves optical networks, thus revealing the design rules and the material composition responsible for these structures. Cryo-SEM minimizes the introduction of artifacts that could alter the geometry of the cell's arrangement and thus affect the living optical network; sections and punched tissues are obtained from leaves, frozen, and imaged in frozen conditions. EDX and Raman determine the material composition (expected are cellulose, lignin, chlorophyll, and anthocyanin dyes) and its effect on the network. Since natural variability might induce differences in the network parameters due to cells belonging to different leaves and plants, statistical methods typical of colloidal crystal lattices are used as part of the analysis and to direct optical modeling (radial distribution function, angular distribution function, Fourier Transform).

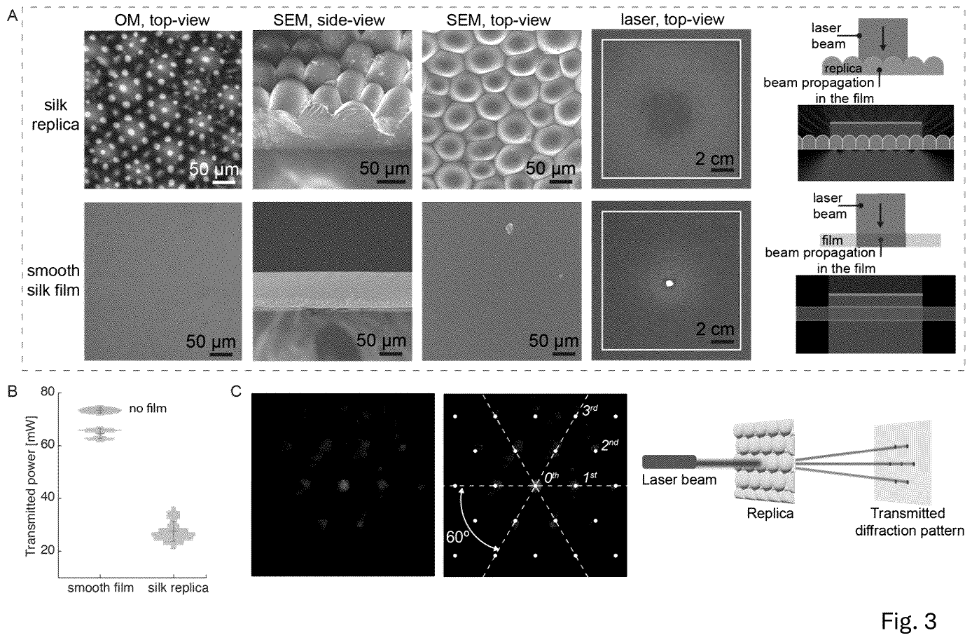

Results—(1) Selected one low-light orchid species, Macodes petola, and one low-light non-orchid species, Begonia rex, and cultivated them at 90% RH and T=25° C. (2) Demonstrated presence of round cells, with uniform diameter (~33 μm±4.9 μm), that are arranged in a short-range order lattice (a living optical network) with hexagonal symmetry on M. petola using optical microscopy and cryo-SEM. B. rex shows round cells, yet arranged in a less ordered lattice. (3) Measured horizontal light coupling within the network in M. petola: this consists in golden stripes/marks running along the green cells in FIG. 3 (central dot is specularly reflected light) when leaves are illuminated with visible light. (4) Measured wavelength-dependent pattern on M. petola: using red laser light (λ₁=633 nm) to illuminate the leaf, the light is seen only along cells with low chloroplast concentration due to selective absorption.

Optical Model to Understand Key Parameters of in-Plane Light Propagation and Photosynthesis Efficiency and Grow Plants Under Various Light Conditions.

An optical model is used to investigate the structure-function relationship of network patterns in light management in a parameter range not necessarily accessible with living plants (e.g., the scale of the network changing from the micro- to the nano- or to the macro-level; Intensity of the light attenuation when the number of elements forming a single network is higher than a few thousand). Similarly, expanding the growth conditions of selected plants affords to explore if the network-facilitated light management can be further optimized in terms of photosynthetic efficiency compared to what observed under the plants' natural growing conditions. This disclosure lays the foundation for harvesting advanced living optical systems essential for the subsequent fabrication of biopolymeric plant-inspired replicas.

Methods—(1) Develop optical models using ray tracing and Finite-Difference-Time-Domain (FDTD) approaches to investigate the cells' arrangement at the microscale and the role of any nanostructure and absorbing elements (chloroplasts) on light interaction, respectively. Ray tracing models determine the key parameters of in-plane and vertical light propagation by primarily evaluating cell shape (flat, concave, convex, conical, or irregular) and cells' arrangement in a lattice (hexagonal or square) for various illumination conditions (flux density ~40-350 PPFD, directed or diffuse light, visible broadband light λ=400-750 nm or single wavelength light k_{blue}~480 nm and k_{red}~633 nm). Additional parameters to study include cell size (diameter ~5 μm-100 μm), polydispersity (low 0.0, medium 0.5, high ~1), and composition (ratio of cellulose/lignin), lattice type and order degree, and lattice curvature (to take into account due to the natural presence of veins along each leaf). Based on the best combination of structural parameters found by the ray-tracing model, the FDTD model is then used to investigate the role of photosynthetic elements concentration (~1-100/cell) and photosynthesis conversion rate (expected 5-10%) within the network and any nanoscale feature that might be found within the networks. The optical models also determine if the studied naturally occurring structures are already optimized for the maximum light harvesting and lateral redistribution or if different combinations of the cells' geometrical arrangement and composition can, instead, lead to a higher photoconversion yield. Running these sets of complementary optical simulations determines the design rules responsible for the observed optical coupling and helps drive the fabrication of the biopolymer replicas. (2) Simultaneously, grow a subset of plants under varying light conditions (e.g., light intensity, directionality, and wavelength range) to control expression of relevant optical features and to quantify light-driven adaptations within the living optical network (e.g., cell shape and size, lattice regularity, photosynthetic molecules concentration and distribution). Light flux density is progressively decreased to reach extremely low flux density (from ~80 to ~0.PPFD with steps of 10 PPFD first and 0.5 PPFD later and as directed by optical simulations for low light plants) to see how the network changes in extremely low illumination conditions (chloroplasts' concentration and cells' convexity should increase). Direct (light normal to the leaf) and diffuse (light reaching the leaf from various angles) light conditions, and single wavelength (λ_{blue}~480 nm and λ_{red}~633 nm) as opposed to broadband (λ=400-750 nm) light conditions are tested for each investigated PPFD range for at least 5 plants/species. Plants' growth is monitored on a weekly basis to assess adaptation to the new illumination conditions and is adjusted depending on plants' response. This growth condition screening allows finding the ideal growth conditions for plants to show the best combination of cells' arrangement and composition to maximize photosynthesis and to direct replica fabrication. (3) Conduct optical analysis and morphological investigation to assess the effect of varying growing conditions and of expected light-driven adaptation. Specifically, the macro- and micro-scale appearance of a statistically significant number of selected leaves (5+/plant) of selected orchids is characterized in vivo at set time intervals (from once a day to once a week depending on species growth rate) to capture and quantify geometrical modification of the optical networks as a function of the varying illumination conditions. Additionally, since plants' exposure to varying illumination conditions is expected to affect both the concentration and the spatial location of the photosynthetic elements (and thus the overall ability and efficiency of the plants to carry out photosynthesis), these parameters are assessed using multi-laser (blue, green) confocal microscopy exploiting the natural fluorescence of photosynthetic elements such as chlorophyll; similarly, spectrophotometer measurements of photosynthetic elements directly extracted from the leaves (in solution) determine the type and concentration of photosynthetic elements for the various investigated growing conditions. These measurements quantify the best conditions for plants to maximize solar energy extraction.

Results—(1) Investigated effect of the cells' shape on the light propagation by using a ray-tracing model: single cells illuminated with white light showed an increase of laterally reflected light upon increase of their convexity. (3) Implemented the model for one row of cells illuminated with white light: by illuminating one single cell of the row, the row made of cells with the highest convexity showed that light propagated the furthest away from the illumination point, thus showing the maximum horizontal redistribution of light. (3) Demonstrated that the lateral redistribution of the light increases upon increase of the angular width of the illumination source for white light. (4) Cultivated one species (M. petola) at 90% RH, T=25° C., with no direct light illuminating the plants; observed chloroplast concentration increase and redistribution.

### Fabricate and Optimize Plant-Inspired Biologically Based Optical Replicas.

The fabrication and implementation of plant-inspired biologically based replicas of the patterns observed in low-light plants is essential for validating the proposed light management mechanism. This disclosure translates a research-based discovery into prototypes applicable in industrial settings for sustainable energy harvesting, offering a platform for innovative light redistribution.

Methods—(1) Fabricate and characterize bio-inspired biologically based free-standing replicas of plants displaying efficient optical networks using biopolymers able to replicate the multiscale patterns present on the leaves with high fidelity. Water-based solutions of regenerated proteins (silk fibroin blends) are used to capture the patterns by casting them on the leaves of living plants grown under illumination, RH, and T conditions that maximize the expression of the living optical network and of the photoconversion efficiency. Silk fibroin is used since it enables high optical clarity (transparency), full water-based processing, bulk doping, it can stabilize labile molecules usually prone to degradation (e.g., chlorophyll) and can conform to and replicate macro-to-nanostructures with high fidelity. Plant-inspired replicas made of silk fibroin reproduce the leaves' microscale pattern with high fidelity. The effect of the following silk matrix parameters on the optical response of the replicas is investigated: silk solution concentration (0.1-10 wt %), film thickness (10-200 μm), and protein conformation (to give flexible amorphous or more rigid crystalline films). The fabricated replicas are characterized through optical microscopy, spectroscopy, and morphological analysis following the methods disclosed herein and the static optical performances are checked against the optical models disclosed herein. These measurements determine the feasibility of fabricating plant-inspired materials that are patterned to display a light-coupling mechanism similar to that observed in plants. (2) Program optical functionality of replicas by tuning biomaterials composition, conformation, cell shape, size, polydispersity, and lattice parameters by using silk-based blends. To do so, introduce chemical functionalization by selective doping with dyes (e.g., fluorescein and rhodamine) to control and direct the light propagation for specific wavelength ranges along the network, mimicking the variety of pigments and their heterogeneous distribution as found in plant leaves. Spatially pattern the replicas to create spatially dependent variations in transparency to regulate the diffusive/scattering optical behavior; this is done through reconfigurable transitions of the optical network (e.g. make the films water resistant by increasing silk matrix crystallinity or water-soluble by keeping silk amorphous). Additionally, deposit thin layers (low density silk blends) to induce total internal reflection to increase the amount of light that can be absorbed by the replicas (increase the network efficiency), These experiments are based on a single main material to fabricate the matrix (silk and its blends) due to its versatility and they enable the fabrication of a variety of systems for light management solutions. Characterize the replicas as described elsewhere herein and optimize their form-factor by comparing their optical performance with natural counterparts and optical models. This set of measurements determines the versatility of the fabricated replicas. (3) Integrate replicas into optical devices to fabricate prototypes including plant-inspired solar cells (patterned silk replicas used as top layers to maximize light collection), reflectors with strain-dependent optical response (film changes from metallic to transparent upon stretching), wavelength-dependent switches (doped replicas transmit single wavelengths since the others are absorbed by pigments), light diffusers (highly opaque patterned replicas), and unclonable security tags (replicas with short-order/disordered arrangement of cells). Fabricating prototypes that use the optical network as the mechanism for light redistribution crucially demonstrates the application opportunities of this novel light management mechanism.

Results—(1) Fabricated positive and negative replicas of one species (M. petola) using water-based protein solution (regenerated silk). (2) Confirmed transfer of the short-range order microscale pattern by measuring the far-field diffraction pattern of replicas which displayed a 6-fold symmetry pattern typical of hexagonal lattice (See Guidetti, G.; Sun, H.; Marelli, B.; Omenetto, F. G. Photonic Paper: Multiscale Assembly of Reflective Cellulose Sheets in Lunaria Annua. Sci. Adv. 2020, 6 (27), eaba8966 and Wang, Y.; Li, W.; Li, M.; Zhao, S.; De Ferrari, F.; Liscidini, M.; Omenetto, F. G. Biomaterial-Based “Structured Opals” with Programmable Combination of Diffractive Optical Elements and Photonic Bandgap Effects. Adv. Mater. 2019, 31 (5), 1805312, each of which is incorporated by reference in its entirety for all purposes.). (2) Measured light coupling in replicas with optical microscopy which showed horizontal bright strikes along each dome. (3) Fabricated replicas doped with fluorescent dye and measured enhancement in fluorescence given by the micropattern. (4) Performed red laser imaging of positive and negative silk replicas and compared to rough and smooth silk films: positive and negative replicas show more homogeneous laser light distribution in the plane of the films. A larger area of the replica films is illuminated with red light and the transmitted light is lower for the replicas (~20%) than for the rough (~60%) and the smooth films (~70%). This demonstrates horizontal light redistribution in the biomaterial replicas.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Examples

Example 1 describes cell-based living optical networks in orchid leaves for light harvesting and manipulation, as may be applicable to embodiments of the present disclosure.

### Surface Area Enhancement

For a spherical dome of radius r and height h, the surface area of the dome is given by: A_{dome}=2πrh (1). The corresponding surface area of the region inscribed by a circle of radius r is given by: A_{circle}=πr² (2). Therefore, the increase in surface area given by the presence of a dome, rather than a flat surface is given by:

$$\frac{{A}_{dome}}{{A}_{circle}} = \frac{2 h}{r}$$

(3). For r_{avg}=30 μm (r_{avg, green}=30.3 μm±5.9 μm, N=269) and h_{avg}=49 μm (h_{avg}=49.6 μm±8.7 μm, N-100), there is a ~A_{dome}/A_{circle}~3.3 increase in area.

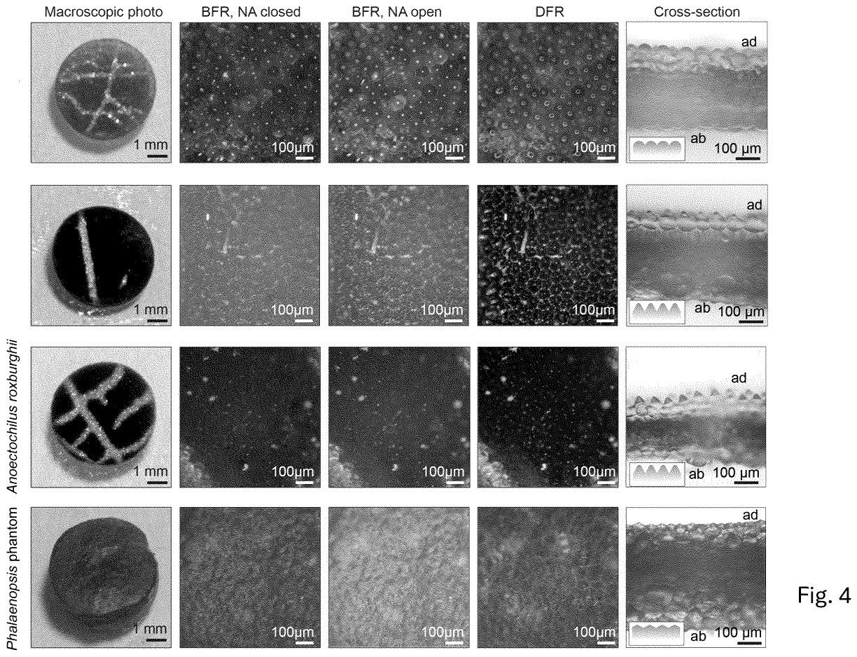

Macodes petola belongs to the genus Macodes Lindl and is commonly recognized as a “jewel orchid” due to its distinctive foliage. This plant typically grows in environments characterized by high humidity, with varying light conditions ranging from brief and intense sunflecks to low and diffused light. Macodes leaves exhibit a visually striking golden net-like pattern on the upper epidermis that alternates with surrounding green and dark green cell domains in the other parts of the leaf (FIG. 1A, B, FIG. 4).

Bright field reflection microscopy shows that both the golden and green regions of the leaf consist of densely packed cells with a round morphology (FIG. 1B-C, FIG. 4). The green appearance, characteristic of most leaves, is attributed to the presence of chlorophyll pigments within the photosynthetic centers of the cells, which absorb light for photosynthetic conversion of solar energy into chemical energy. Cryo-scanning electron microscopy (cryo-SEM) of a portion of the leaf confirms that there are densely packed cells which locally appear to have a six-fold arrangement (FIG. 1D-F). The epidermal cells exhibit a distinctive round cross-section (FIG. 1E), in contrast to the conical or more typical flat-top morphology observed in the leaves of the majority of orchid species (FIG. 4). The dome-like shape of the upper epidermal cells in Macodes petola provides also a ~3.3-fold increase in surface area compared to cells with a flat top of the same radius; this unique morphology could offer a potential advantage in capturing more light, given the increased area exposed to light.

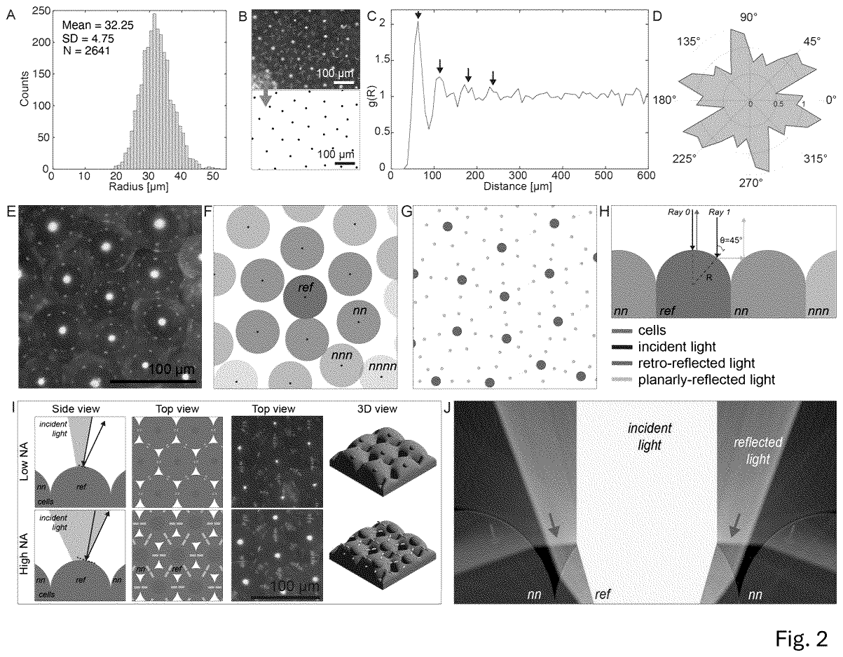

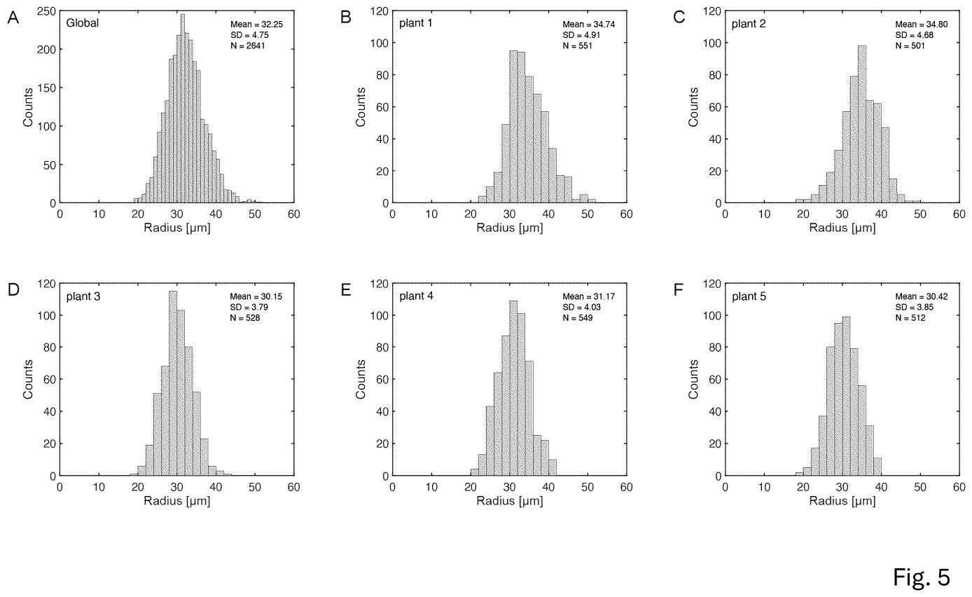

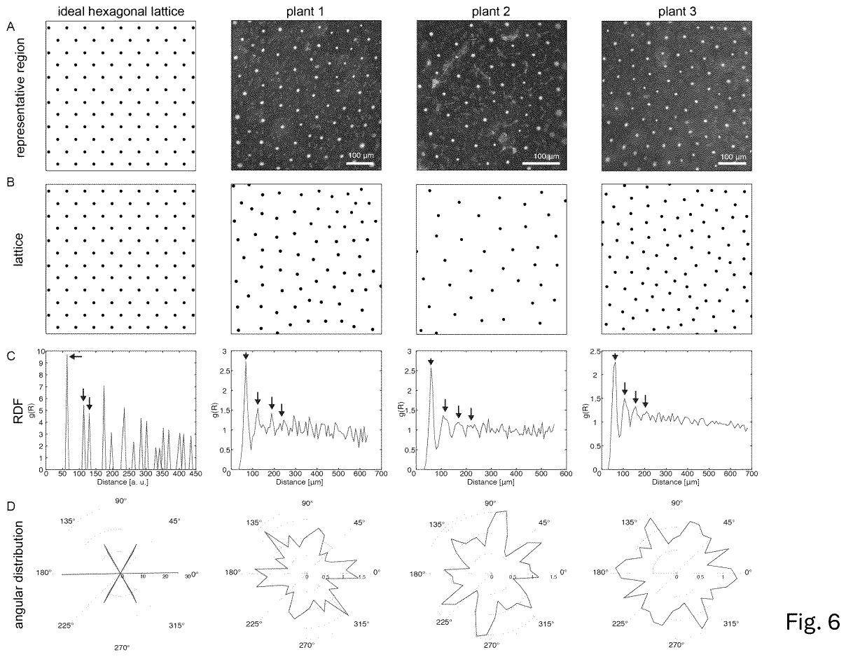

When the leaf is illuminated, a reflected pattern made by an array of bright dots appears overlayed to each cell (FIG. 1C). Optical microscopy of the leaf's upper epidermis demonstrates the consistency in cell size (FIG. 2A, FIG. 5): statistical analysis shows that cells exhibit low polydispersity (p=0.15) and an average radius of r_{avg}~32 μm±4.7 μm (FIG. 2A, FIG. 5). The cells also display tight and seemingly regular packing (FIG. 2, FIG. 6), suggesting a non-random arrangement of the cells. This is further investigated by evaluating both the radial and angular distribution functions of the epidermal cells of the leaf Specifically, the centroid position associated with each cell was extrapolated from microscope images of the Macodes leaf section to generate a “leaf lattice” (FIG. 2B), which was then used to compute the actual leaf's radial distribution (FIG. 2C) and angular distribution function (FIG. 2D). This allows the evaluation of the cellular order found on the leaf's surface in comparison to an ideal hexagonal lattice (Figure S3). The statistical analysis reveals the presence of distinct peaks in the radial distribution function at short distances from an arbitrary chosen reference cell. These peaks disappear when longer distances are considered, indicating the presence of a short-range order in the Macodes cell assembly. Moreover, the angular distribution function reveals the presence of six main lobes arranged at approximately 60° from each other, consistent to what is observed for an ideal hexagonal lattice (FIG. 6). While there is a deviation from the symmetry observed in the ideal case (FIG. 6) visible from broader peaks and small axial deviations, the statistical analysis provides convincing evidence for the existence of short-range order and hexagonal array assembly of the upper epidermal cells in Macodes petola leaves (FIGS. 2B, C, and D, FIG. 6). Although no long-range order is apparent, as expected given the inherent variability of natural tissues, the epidermal cells in Macodes leaves exhibit a higher order degree compared to other jewel orchid species and compared to common orchids (FIG. 4).

The illumination of an ideal hexagonal lattice made of perfectly round refractive elements causes a portion of the light to retroreflect and another portion to be reflected laterally to other elements before being reflected away from the elements normally (FIG. 2H). This process generates 6 lateral reflection spots (also known as cross-communication spots) on each nearest neighbor refractive element (nn) surrounding the illuminated central reference element (ref); consequently, this results in the horizontal redistribution of perpendicularly incident light within the plane of the lattice. This mechanism of geometry-driven light redistribution can be described as an optical network since the individual elements when taken collectively can manage light and redirect it to different portions of the elements' assembly. Notably, the phenomenon of lateral light redistribution has been documented for self-assembled optical networks formed by spherical microdroplets of cholesteric liquid crystals, bottlebrush block-copolymers, and raspberry-like colloids, but has not been observed in natural systems, potentially due to the limitations imposed by the shape, size, and packing of cells across different species.

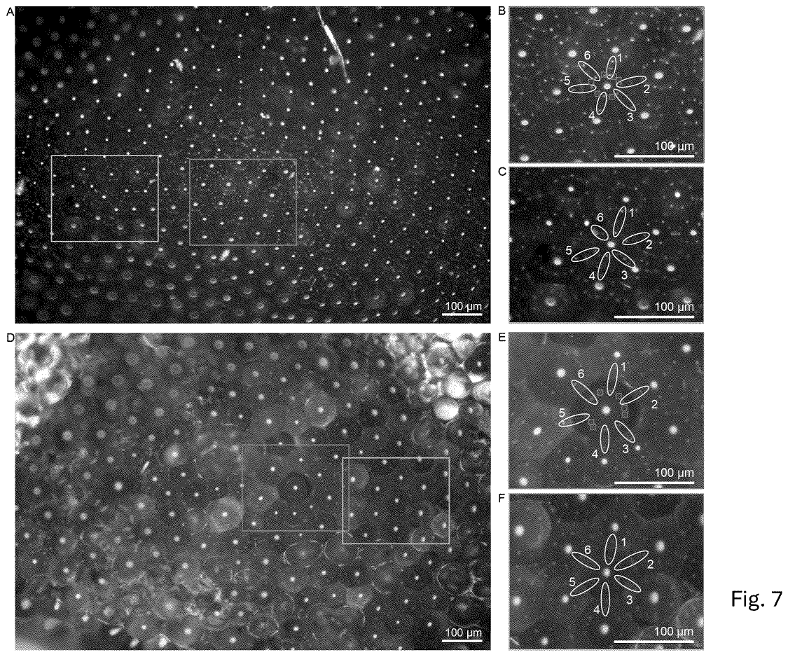

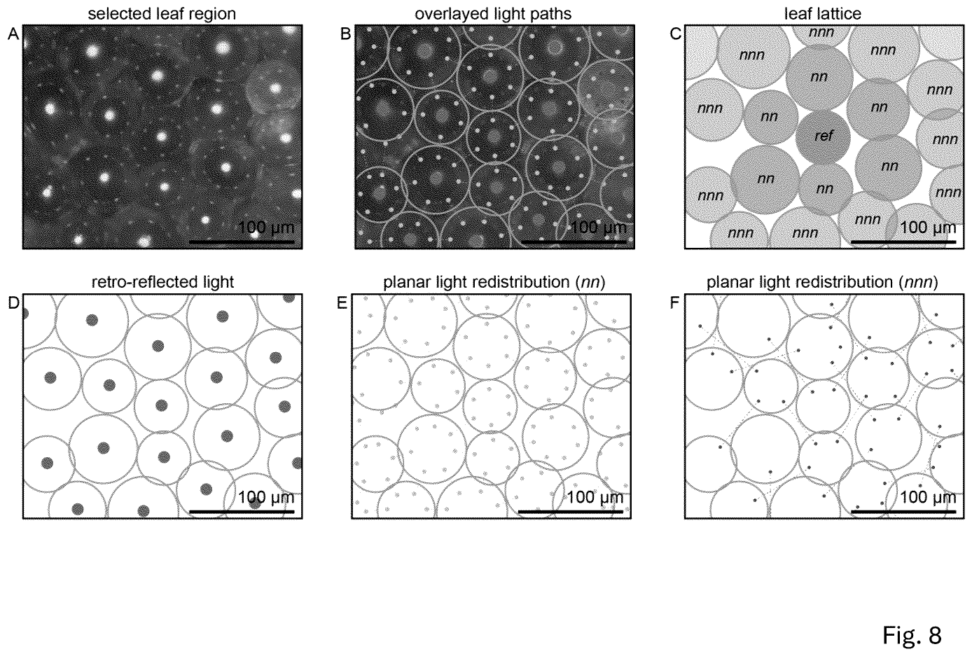

In Macodes leaves, a convergence of three key morphological and geometrical factors takes place, specifically (i) the rounded morphology of the cells (FIG. 1), (ii) their low size polydispersity, and (iii) their arrangement in a short-range order hexagonal lattice (FIG. 2C, D, FIG. 6), giving rise to a distinctive network-like optical response. When a Macodes leaf is illuminated with visible light, a discernible short-range order pattern with 6-fold symmetry, characterized by bright reflection spots, emerges across the cell network on each cell's top and sides (FIG. 1C, 2E, FIG. 7, FIG. 8). Specifically, when a cell (ref cell in FIG. 2F, FIG. 8) is illuminated, a portion of the incident light is reflected horizontally within the plane of the leaf onto neighboring cells (nearest neighbor nn in FIG. 2H, FIG. 8). This generates the bright reflection spots observed on the sides of the nn epidermal cells (FIG. 2E, 2G); cross-communication spots between a ref cell and next nearest neighbor nnn cells are also visible in most regions (FIG. 8). This cross-communication reflection pattern aligns with mechanisms of light redistribution previously documented in self-assembled optical networks formed by spherical microdroplets, thus indicating the formation of a living optically coupled network within the leaf structure. Through this network, cells efficiently redistribute light laterally between adjacent domains in the plane of the leaf (FIG. 2E-H). Despite the low polydispersity observed, the inherent natural variability in cell morphology and packing within the Macodes leaves leads to additional in-plane coupled reflections and refractions of the light. This is attributed to the larger number of cells that can directly face each other, resulting in increased cell-to-cell light coupling. This is evident in the higher number of lateral reflection spots, that in some portion of the leaves can reach up to 12 (FIG. 7).

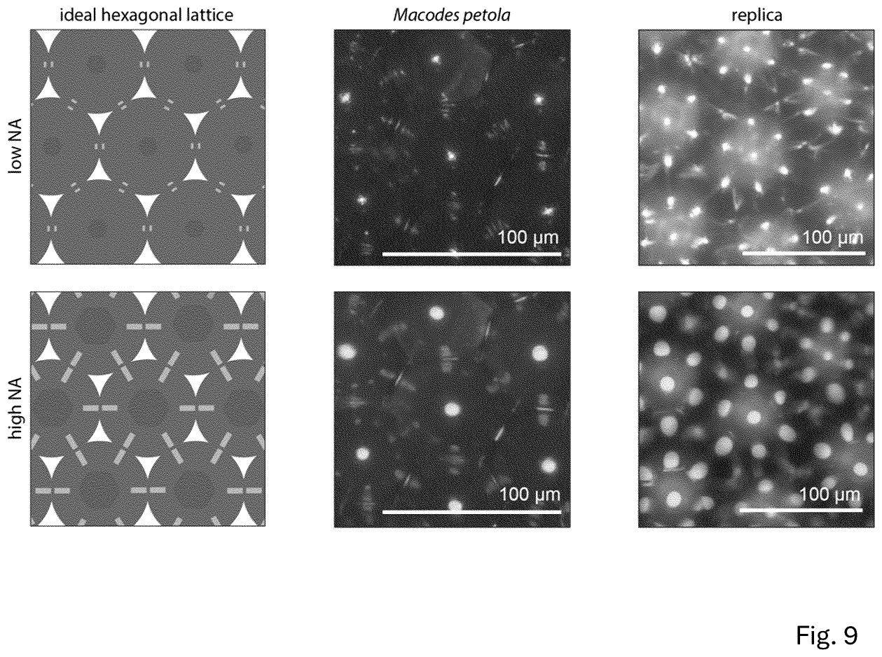

The cross-communication between cells is strongly dependent on the illumination conditions, specifically, it is directly proportional to the angular distribution of the cone of incident light (FIG. 2I). Consequently, this variation influences the amount of light propagated laterally through the cell network (FIG. 2I, FIG. 9). This implies that when the Macodes leaf cells are studied through an optical microscope, larger retroreflection and larger cross-communication spots are observed for progressively larger numerical apertures (illumination cones).

Light redistribution within the cellular array was modeled using ray tracing (FIG. 2J). When cells are arranged in a closed-packed lattice, a fraction of the refracted light is redirected on neighboring cells, subsequently undergoing additional reflection and refraction within those cells (and their neighboring ones). This lateral light redistribution mechanism facilitates light guiding from a directly illuminated cell to a cell not directly exposed to the original illumination, thus maximizing light redistribution along the plane of the leaf. Not only does this arrangement facilitate the concentration of a portion of the light within the leaf in the z direction because of each individual refracting element, but it also enables horizontal light distribution through the cell collective behavior. Although the precise functional significance of this distinctive cellular arrangement in Macodes petola jewel orchid leaves requires further investigation, the observed lateral light redistribution of incident light suggests a potential strategy employed by the plant which could involve mitigating the impact of intense direct light rays by dispersing them across multiple cells, thereby avoiding concentration on a small volume of chloroplasts and associated photoinhibition and photodamage.

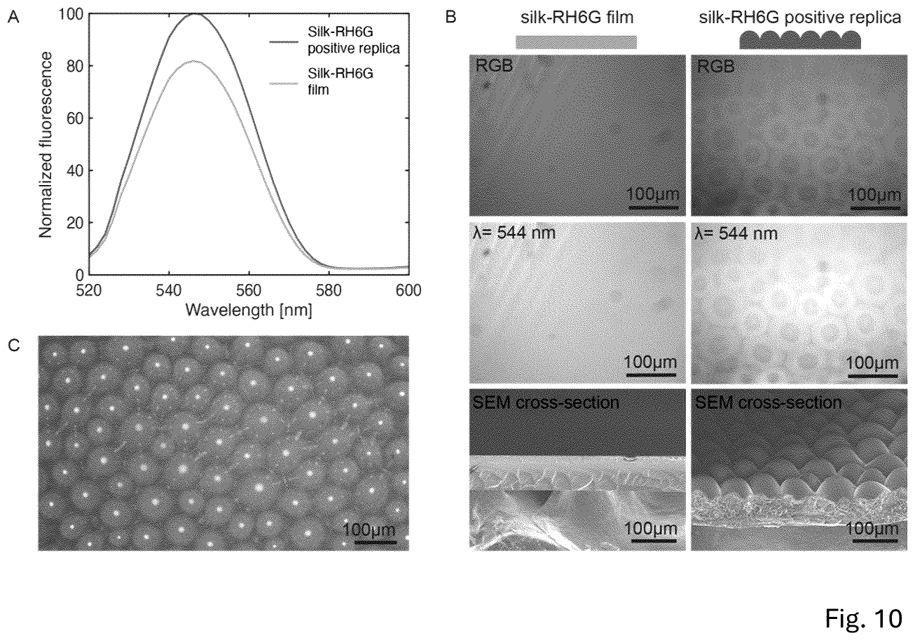

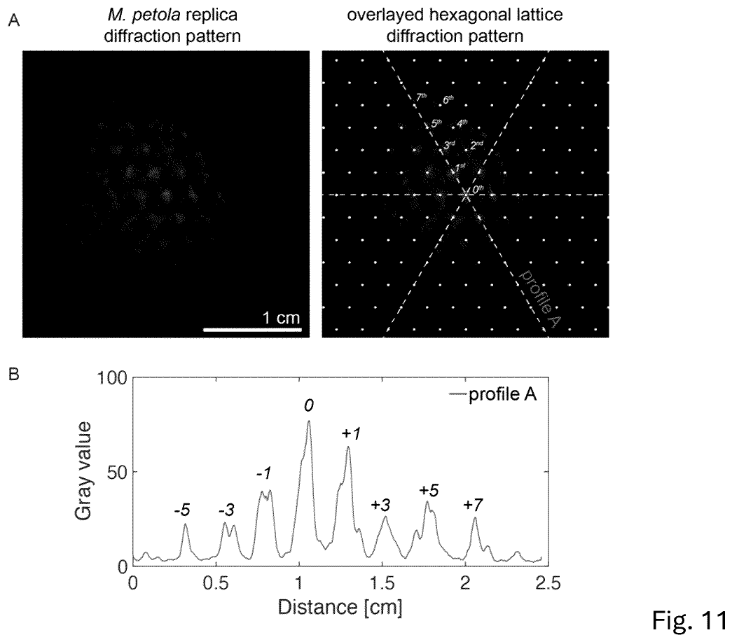

To further investigate the unique Macodes leaf microscale patterning, its structure was replicated using silk fibroin and leveraging silk's ability to conform and replicate micro-to nanostructured surfaces with a fully-water based process that maintains high fidelity and optical clarity (See the following publications, each of which is incorporated by reference in their entirety for all purposes: B. D. Lawrence, M. Cronin-Golomb, I. Georgakoudi, D. L. Kaplan, F. G. Omenetto, Biomacromolecules 2008, 9, 1214.; Y. Wang, B. J. Kim, B. Peng, W. Li, Y. Wang, M. Li, F. G. Omenetto, Proceedings of the National Academy of Sciences of the United States of America 2019, 116, 21361.; Y. Wang, D. Aurelio, W. Li, P. Tseng, Z. Zheng, M. Li, D. L. Kaplan, M. Liscidini, F. G. Omenetto, Advanced Materials 2017, 29, 1.; Y. Wang, B. J. Kim, G. Guidetti, F. G. Omenetto, Small 2022, 18, 2201036.; S. Kim, A. N. Mitropoulos, J. D. Spitzberg, H. Tao, D. L. Kaplan, F. G. Omenetto, Nature Photonics 2012, 6, 818.; G. Guidetti, L. D'Amone, T. Kim, G. Matzeu, L. Mogas-Soldevila, B. Napier, N. Ostrovsky-Snider, J. Roshko, E. Ruggeri, F. G. Omenetto, Applied Physics Reviews 2022, 9, 011302.; G. Guidetti, Y. Wang, F. G. Omenetto, Nanophotonics 2020, 10, 137.) A thin layer of PDMS was applied directly onto the surface of the leaf of a living plant, successfully replicating its structure and forming a negative replica of the Macodes leaf By casting silk on the PDMS mold, a free-standing silk positive replica is obtained. The silk-leaf faithfully reproduced the round morphology of the cells observed on the plant, their short-range order hexagonal pattern, and their distinctive light coupling mechanism through transversal cross-communication spots (FIG. 3, FIG. 9, FIG. 10, FIG. 11).

The Macodes leaf replicas were characterized both by optical microscopy (OM) and scanning electron microscopy (SEM) while their optical response was compared to smooth unpatterned silk films (FIG. 3A). The replicas displayed cross-communication pattern among neighboring cells when illuminated with visible light, similarly to what observed in the plant leaves. Imaging (FIG. 3) shows the microscale pattern formed by round refractive elements that are tightly packed and are responsible for light redistribution in the leaf, which can be further modulated by varying the illumination conditions (in contrast to smooth silk films as shown in FIG. 9).

The optical performance of the silk replicas is assessed by illuminating them with laser light through the films and comparing the transmission distribution plots with those of smooth, unpatterned silk films (FIG. 3A, B) (See G. Guidetti, Y. Wang, F. G. Omenetto, Nanophotonics 2020, 10, 137., which is incorporated by reference in its entirety herein for all purposes). Unpatterned silk films exhibited low attenuation and scattering. In contrast, the patterned replicas show a substantial transversal broadening of the laser spot-size, suggesting an enhancement in the planar redistribution of light from the collective optical behavior of the cell assembly. By doping silk films and silk replicas with a dye (Rhodamine 6G), (FIG. 10) it is possible to see an enhancement of the fluorescence from the replicas, consistently to an increased interaction between the light and the fluorophore. Additionally, the beam intensity distribution was found to cover a larger area (larger than a few cm²) than the typical beam size and its profile seen in unpatterned, smooth silk films. Finally, light was also strongly attenuated as its intensity was reduced by approximately half after propagating through the silk replicas (FIG. 3B).

Light transmission through the clear leaf replicas displays a distinct far-field diffraction pattern that is associated with the short-range order hexagonal lattice (FIG. 3C, FIG. 11). Remarkably, diffraction spots up to the 7^{th} order (calculated with respect to the distance from the 0^{th} order/reference cell) were visible. This observation is consistent with the presence of an ordered micropattern in the silk films and, consequently, in the epidermal cells of Macodes leaves.

Conclusion—While at first glance, the Macodes petola orchid's leaves have an alluring optical appearance, with sparkling golden veins and dark green regions, its true distinction is found in the functional network formed by the leaf cells that is able to capture and redistribute light throughout its surface. This “living optical network” comes from an unusual confluence of regularly sized epidermal cells, their short-range hexagonal packing and, in contrast to many other orchids species, their distinctive rounded cellular shape, transforming the leaf s soft, curvilinear surface into a system with the ability to distribute light across the leaf. It can be speculated that the function of this living optical network could relate to the plant's need to maximize the use and distribution of light in difficult growing environments (such as areas of low illumination,) perhaps leading to increased photosynthetic efficiency through optical management of incoming light.

Such plant-based living optical networks could serve as a source of inspiration for the design of functional materials that efficiently harvest, manipulate, and process light with living optical networks material formats that are soft, conformal, and sustainable, as preliminarily demonstrated by replicating the living structure of the leaf. Living systems such as the one presented here serve as captivating examples of how natural optical networks can offer valuable insights for creating new bioinspired material design strategies for light management with innovative, living optical networks that are adaptive and resilient with possible envisioned utility in next-generation flexible, conformal, light-harvesting and photoconverting skins.

Materials and Methods—Materials: Living specimen of Macodes petola, Ludisia discolor, and Anoectochilus roxburghii jewel orchids were commercially acquired and grown in a custom-made greenhouse under controlled humidity (RHₘᵢₙ=87%, RHₘₐₓ=98%) and temperature (Tₘᵢₙ=17° C., Tₘₐₓ=25° C.). Illumination was provided using 10W full spectrum lights (Barrina, T5) with a cycle of 12 h on and 12 h off. Living specimen of the common orchid Phalenopsis phantom species were commercially acquired and grown at RH_{avg}=12% and T_{avg}=23° C. For each studied species, leaf cross sections were obtained with a hand-held microtome. Rhodamine 6G was purchased from Sigma Aldrich.

Silk solution: Silk solution was prepared by sieving silk fibroin powder (obtained from Bombyx mori silkworm cocoons) through a 300 μm sieve and by dissolving it in double distilled water. The obtained solution was then centrifuged at 10,200 rpm for 20 min at 4° C. (Beckman Coulter, Allegra X-14) and the clear supernatant was then collected, filtered, and stored at 4° C. The solution concentration was 8.48 wt %.

Silk films fabrication: Unpatterned silk films were obtained by casting silk fibroin solution (C=8.48 wt %) in Petri dishes and by letting it dry at RT. Silk positive replicas were obtained by first casting a PDMS solution directly on the leaf, letting it dry, peeling it off, and by using it as a mold to cast silk solution (C=8.48 wt %) in it and let it dry at RT. Silk films with Rhodamine 6G were fabricated by casting the silk-rhodamine solution (C_{rhodamine}=0.1 mg/ml) either in polystyrene Petri dishes or on the PDMS negative mold to give either smooth or patterned films, respectively.

Plant photographs: Digital macroscopic images of the plant specimen were taken with a smartphone (iPhone 11 Pro, Apple) and with a DSLR (digital single-lens reflex) camera (Canon Rebel EOS-Tli) adjusted for exposure and contrast.

Optical microscopy and spectroscopy: Optical microscopy was performed on a customized Olympus upright Olympus BX51 microscope coupled with a DSLR (digital single-lens reflex) camera (Canon Rebel EOS-T1i) using a halogen lamp (U-LH100L-3) as a light source in Koehler illumination. Bright field reflection images were collected using 10× (Olympus, MPlan N, NA=0.25), and 20× (Olympus, LUCPlanFL N, NA=0.45) objectives.

Lattice analysis: Lattice analysis of Macodes petola cells was carried out using the software ImageJ and Corderly. Cells radius and the corresponding centroids position were extracted from bright field reflection images using ImageJ; specifically, the leaves of 5 different plants were examined to extract the radius of N=2641 green cells. The centroid position was then used to calculate the radial distribution function (RDF) and the angular distribution function. These functions were then used to assess the order degree of the lattice.

Scanning electron microscopy: Imaging of the silk positive replicas was performed by mounting the free-standing replica films on aluminum stubs using carbon tape to be either at 90° (top view imaging) or at 0° (cross-sectional imaging) with respect to the electron beam. To ensure electrical conductivity, the stubs were sputtered with ~10 nm of chromium using a Quorum Q150T ES Plus sputter coater. The specimens were imaged using a Zeiss Sigma 300 Field Emission Scanning Electron Microscope (FE-SEM) with an InLens electron detector at 5 kV with 2 to 2.9 mm as the working distance.

Cryo-scanning electron microscopy: Cryo-scanning electron microscopy imaging of the fresh Macodes leaves was carried out as it follows. A small portion of the leaf was isolated using a surgical punch, mounted on a cryo holder and quenched in liquid nitrogen. The leaf was maintained under cryogenic conditions and cut with a blade within a Quorum Chamber before being sublimed and subsequently coated with a thin platinum layer (t ~10 mm) before being transferred to a Zeiss Sigma 300 SEM operating with a secondary electron at 3-10 kV and 2-3 mm as working distance under cryogenic conditions.

Diffraction patterns: The Macodes silk positive replica were illuminated by a red laser (HRR015-1, P=1.5 mW at λ=633 nm, Thorlabs) positioned normally with respect to the patterned replica. The transmission diffraction patterns were collected on a white screen positioned normally with respect to the samples and captured using a digital single-lens non-reflex camera (Canon, EOS M50) located in front of the screen. The software ImageJ was used to analyze the patterns.

Laser light propagation in silk films: The silk films were illuminated by a red laser (λ=650 nm, Shenzhen Yize Technology Co., XS09) positioned normally with respect to the films at a distance d₁=7 cm. The transmission patterns were collected on a screen positioned at a distance d₂=11 cm away from the samples and captured using a camera (Canon Rebel EOS Tli) located at the back of the screen. The attenuation of the laser beam caused by the interaction with the silk films was measured using a power meter (Thorlabs, PM100A) equipped with a thermal power sensor head (Thorlabs, S310C) placed at a distance d₂=13 cm away from the laser. The silk films were positioned at a distance d₃=12 cm from the laser.

Optical modelling: Light interaction with the cells in the upper epidermis of the leaves was simulated using the phydemo.app software for ray tracing simulations.

Fluorescence imaging: To perform fluorescence analysis of the silk films doped with Rhodamine 6G and of the silk positive replicas doped with Rhodamine 6G, an Olympus IX71 microscope equipped with a mercury lamp as a light source was coupled to a multispectral camera (CRI Nuance Ex). The films were observed using a 10× objective (Olympus, UPlan FL N, NA=0.3) in bright field fluorescence. The spectral maps were acquired in the range λ=520-600 nm with a step size of 5 nm, using an excitation filter (Ex=480/40 nm) to filter the incoming light. The software Nuance 3.0.2 was used to acquire and unmix the maps in different spectral components and the software MATLAB R2023a was used to analyze the data.

Example 2 describes cell-based living optical networks in orchid leaves for light harvesting and manipulation, as may be applicable to embodiments of the present disclosure.

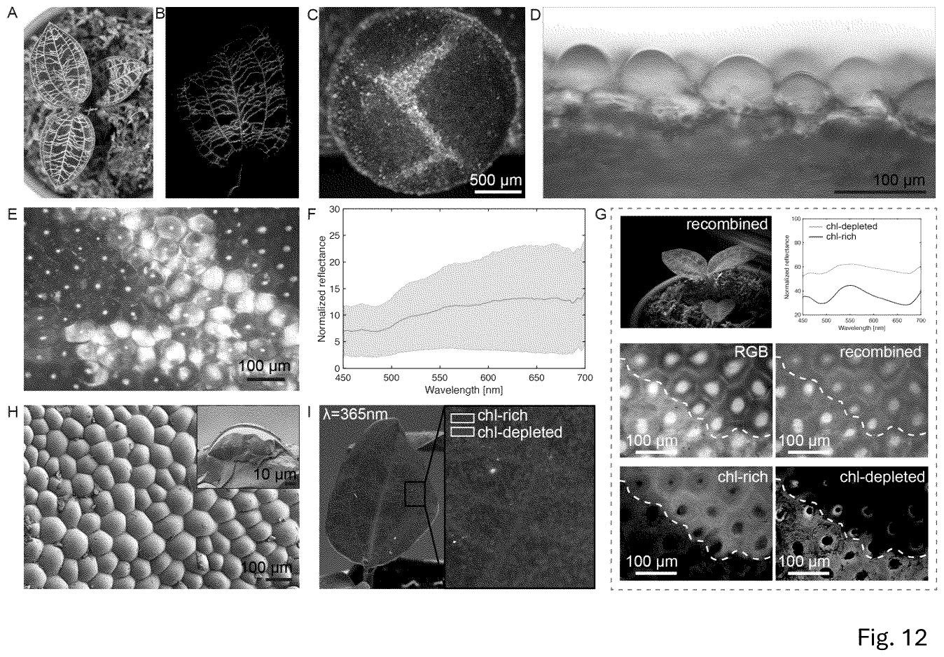

Macodes petola is a plant belonging to the genus Macodes Lindl and commonly attributed to the family of the “jewel orchids” due to its distinctive foliage; it is found in moist and mossy locations characterized by high humidity and generally low diffused light. Macodes leaves exhibit a striking reflective reticulated pattern on the upper epidermis that appears overlayed to the vein network (FIG. 12A, B). Macroscopically, the vein pattern appears strongly metallic, with a golden reflection that is accentuated by the surrounding green and dark green domains in the other portions of the leaf.

Bright field reflection microscopy shows that both the golden and the green regions of the leaf are formed by a series of tightly packed cells with round morphology (FIG. 12C-E). The green optical appearance typical of most leaves is caused by the presence of chlorophyll (chl) pigments which are found within the photosynthetic centers of the cells (chloroplasts) and absorb light to photosynthetically convert it to chemical energy. At higher magnification, the individual cells located within the veins appear brightly reflective unlike the nearby cells (FIG. 12E, F). This seems to indicate that the upper epidermis of the Macodes leaf is formed by specialized cells which are either green and rich in chlorophyll (chl-rich) or highly reflective and with negligible amounts of chlorophyll (chl-depleted), as also confirmed by spectral analysis of the leaf (data not shown). Multispectral imaging of both the plant and the leaf shows the spectral separation between chl-rich and chl-depleted cells both macroscopically and microscopically, with the reticulated pattern shown on every leaf and on selected portions of the leaves (FIG. 12G). The average reflectance spectrum collected from the chl-depleted cells has broader bandwidth than the average reflectance spectrum of the chl-rich cells, consistently with their observed metallic and green optical appearance, respectively (FIG. 12F, G). The heterogeneous distribution of chlorophyll is also confirmed by UV imaging with ch-rich cells displaying strong red fluorescence and chl-depleted cells displaying no signal in correspondence of the leaf veins (FIG. 12I).

The distinctive metallic look of the leaf veins is angle-dependent, yet microscopic analysis of the reflective domains shows only limited reflectance and transmission in the normal direction (FIG. 12F), suggesting that the incident light is redirected over a broad range of angles and not simply focused by the cells into the leaf's photosynthetic centers as previously reported for leaf upper epidermis cells.

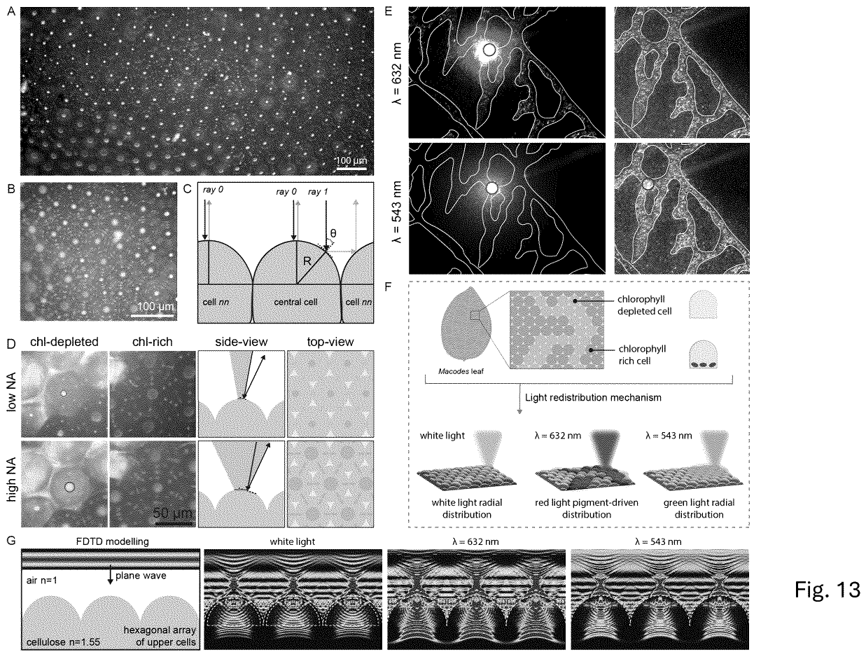

Cryo-scanning electron microscopy (cryo-SEM) of a portion of the leaf shows tightly packed round cells that display a hemispherical cross-section (FIG. 12H), unlike the more common conical morphology observed in other jewel orchid or in common orchid leaves species. The dome-like shape of the upper epidermal cells provides a ~3.3 increase in the surface area compared to a cell with the same radius but flat, thus suggesting that the cells' morphology could aid in capturing more light due to the increased area exposed to light. Optical microscopy of the leaf's upper epidermis shows high regularity in the cell size (data not shown) and tight packing of the hemispherical chl-rich and chl-depleted cells that form a lattice with short range order (FIG. 13). As common for plant tissues, no long-range order of the lattice can be observed, yet the Macodes leaves show higher order degree compared to other species of jewel orchid displaying a reticulated pattern and compared to common orchids (data not shown). The round shape of these epidermal cells combined with their low polydispersity (r_{avg}~30 μm) drives the formation of a hexagonal lattice with 6-fold symmetry (FIG. 13A, B), unlike the typical cells' arrangement observed in more commonly found orchid species.

When the leaf is illuminated with visible light, a high order reflection pattern is noticeable throughout the cell network (FIG. 2A, B), with the leaf displaying 6-fold symmetry reflection features among its cells, suggesting that the leaf structure forms an optically coupled network where cells exchange light between adjacent domains. This in-plane light redistribution mechanism arises from the convergence of cells having round shape, regular size, and packing in a short-range order hexagonal lattice. The observed reflection pattern is consistent with previously observed light redistribution mechanisms in self-assembled optical networks formed by spherical droplets of cholesteric liquid crystals but not yet documented in natural systems due to the geometrical constraints of cells shape, size, and packing.

Specifically, when a leaf cell is illuminated, because of its morphology and its packing, a portion of the incident light can be reflected horizontally in the plane of the leaf onto a neighboring cell. For a hexagonal lattice made of perfectly round particles, this would result in 6 fundamental lateral reflection spots on each cell and in the horizontal redistribution of light within the plane of the lattice, perpendicularly to the incoming direction (FIG. 13C). The limited, yet measured natural variability of the cells morphology within Macodes leaves, causes additional light redistribution within the plane of the leaf due to the larger number of lateral cells regions directly facing each other, resulting in increased cell-to-cell light coupling, which is manifested with a higher number (up to 12) of lateral reflection spots. The observed light network between the cells is strongly dependent on the illumination conditions which, in turn, vary the amount of light that is propagated laterally through the cell network (FIG. 13D). Specifically, the cross-communication reflections spots area is directly proportional to the angular distribution of the cone of incoming light. The optical coupling mechanism in the upper epidermis of the Macodes leaf short-range order lattice appears to be the same for all cells, regardless of the chlorophyll distribution (FIG. 13D).

The spherical shape of the upper epidermis cells non only enables focusing a portion of the light within the leaf in the z direction, but also enables strong specular reflectance and horizontal light distribution which account for the velvety and shiny optical appearance of the leaves. Finite-Difference Time-domain (FDTD) modelling of light interaction with a hexagonal array of spherical cells compared to an array of conical cells or to a flat film show that the shape of the cells drives the strong specular reflection observed on the top part of the cells, while the regular arrangement in a short-range order lattice enables the lateral redistribution of the light (data not shown).

The light propagation in the plane of the leaf was further investigated by illuminating the lattice with a laser (FIG. 13E). Upon illumination of the leaf with photosynthetically relevant radiation (λ=632 nm), the chl-rich cells absorb the light, while the chl-depleted cells display strong red reflectance, thus suggesting that they redistribute the incoming light laterally. This selective red reflectance of the chl-depleted cells results in the guiding of the light away from the initial focusing point, along the metallic veins through cross-communication mechanism. Conversely, light with wavelength outside of the photosynthetically relevant range (e.g., λ=543 nm) is more uniformly diffused through the lattice, without regions of strong selective reflectance. This pigment-mediated light redistribution mechanism (FIG. 13F) suggests a functional strategy for the plant to optimize light delivery to various regions of the leaf not directly illuminated through cell cross-communication within the leaf. FDTD modelling further confirms that the lateral redistribution of light through cross-communication is not only geometrically but also wavelength dependent, with the reflection of photosynthetically-relevant radiation being enhanced compared to non-photosynthetically relevant one (FIG. 13G).

The cell morphology and their arrangement in a short-range order provide the network for in-plane light propagation in the leaf, while the lack of chlorophyll within the reticulated vein cells enables broadband reflectance upon illumination with white light, which would be otherwise suppressed. Traditionally, the lack of chlorophyll in cells would results in a brilliant white appearance; in the Macodes leaves the round morphology and tight packing of the epidermal cells redistributes the incoming light laterally, before being reflected normally, away from the leaf. The cells lattice is curved in correspondence of the veins, increasing the angular distribution of the reflected light and making them appear metallic to the naked eye.

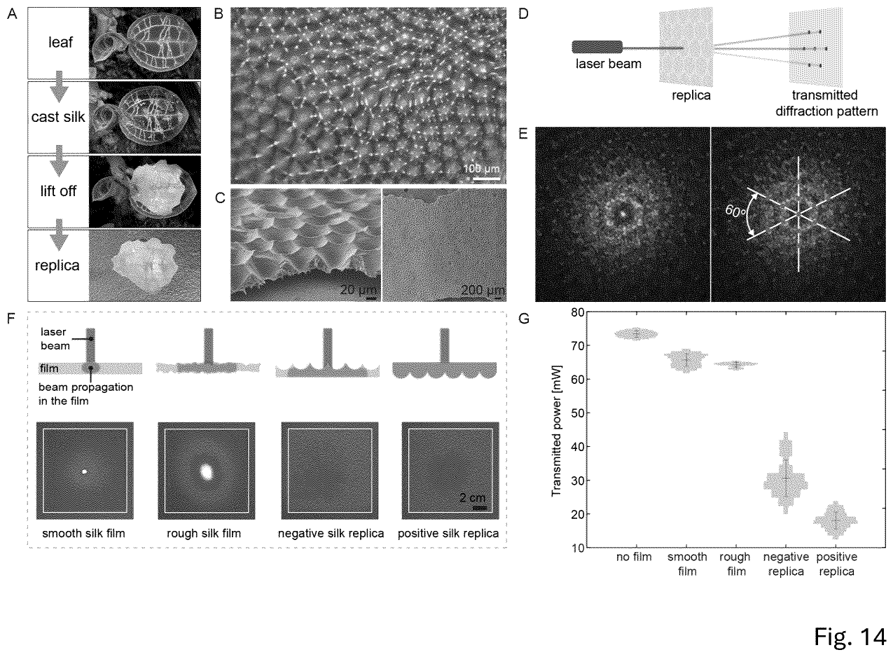

The Macodes leaf upper surface was replicated by casting a thin layer of silk fibroin solution directly on the leaf of a living plant given silk's ability to conform and replicate nanostructured surfaces with high optical clarity (FIG. 3A) (See Lawrence 2008, Wang 2019, Wang 2017, Wang 2022, Kim 2012, Guidetti 2022, and Guidetti 2020). Bright field reflection microscopy and electron microscopy of the so-obtained free-standing silk replica show the short-range hexagonal pattern and the round morphology of the cells as observed on the leaf (FIG. 14B, C). Light propagation through the replicas results in a far field diffraction pattern (FIG. 13D, E) associated to the lattice regularity of the replica, displaying a central peak surrounded by 6 other maxima.

The effect of the lattice on light propagation in the replicas was investigated by comparing the transmission and the attenuation of laser light (λ=650 nm) while illuminating silk films with different surface features (FIG. 14F, G). Low attenuation and minimal scattering of the laser beam is observed through smooth silk films. Expectedly, in-plane scattering increases for increased surface roughness, with associated higher light attenuation and scattering. When light propagates through the Macodes leaf free-standing replica films (both negative and positive) a substantial spread of the laser beam is observed, indicating that the laser light is first redistributed in the plane of the film by the optical network before being transmitted through the film, as also confirmed by the distribution of emitted light from positive silk replicas doped with a fluorescent dye (data not shown). Also, the beam intensity distribution is homogeneous over a few cm², in contrast with the in-plane radial attenuation observed for smooth and rough silk films. The laser power is also strongly attenuated as its intensity is reduced by (approximately) half after propagating through the silk replicas.

Functionally, it has been suggested that such reticulated structures are an evolutionary response of plants aimed at deterring animals either by creating aposematic coloration or through a dazzling optical appearance. The role of metallic veins within reticulated leaves promoting photosynthetically-relevant in-plane light redistribution at the macroscale could also suggest a functional role in redirecting photons towards the chl-rich cells which are not directly illuminated by light. They also seem to provide in plane light distribution that complements the light that the chl-rich cells would receive from perpendicular illumination alone.

Conclusion—In summary, the Macodes petola's striking optical appearance is accompanied by a sophisticated living optical network composed by cells that manipulate and transfer light through their shape and their arrangement in a short-range order hexagonal lattice on the plant's leaves. The heterogeneous distribution of photosynthetic absorbers within the cells alongside the multiple coupled reflections between them result in the characteristic metallic appearance of the vein within the reticulated leaves. The latter also act as wavelength dependent conduits for light redistribution throughout the leaf surface, suggesting an intercellular transport mechanism for photons which could deliver light to cells that are located away from the vein network of the leaf and its metallic domains. Simple replicas of the leaf surface can recapitulate the observed behaviors resulting in a free-standing film with an integrated optical network.

This cooperative optical coupling mechanism across the cell network of the leaf could provide an important means of distribution of light within the structure of the leaf itself given that such plants grow in relatively dark and humid conditions under the tree canopy. These results further confirm that the solutions adopted by nature to manage photons for species survival continue to serve as inspiration for the design of functional materials for light management and processing with utility in new formats of soft, conformal, living optical networks.

Materials and Methods—Materials. Living specimen of the Macodes petola and Anoectochilus roxburghii jewel orchid were commercially acquired and grown in a custom-made greenhouse under controlled humidity (RHₘᵢₙ=87%, RHₘₐₓ=98%) and temperature (Tₘᵢₙ=17° C., Tₘₐₓ=25° C.). Illumination was provided using 10W full spectrum lights (Barrina, T5) with a cycle of 12 h on and 12 h off Living specimen of the common orchid Phalenopsis phantom and of Ludisia discolor species were commercially acquired and grown at RH_{avg}=12% and T_{avg}=23° C. Macodes leaf cross sections were obtained by using a hand-held microtome. Silk powder was provided by Canon CVI and Rhodamine 6G was purchased from Sigma Aldrich.

Silk solution. Silk solution was prepared by sieving silk fibroin powder (obtained from Bombyx mori silkworm cocoons) through a 300 μm sieve and by dissolving it in double distilled water. The obtained solution was then centrifuged at 10,200 rpm for 20 min at 4° C. (Beckman Coulter, Allegra X-14) and the clear supernatant was then collected, filtered, and stored at 4° C. The solution concentration was 8.48 wt %.

Silk films fabrication. Silk films were obtained by casting silk fibroin (C=8.48 wt %) in polystyrene Petri dishes or in polystyrene weight boats and by letting them dry at RT. Casting in Petri dishes was used to fabricate transparent smooth films (smooth silk films) while casting in weight boats afforded rougher and opaque films (rough silk films). Replicas of the leaf pattern were fabricated by casting the silk fibroin solution directly on a leaf of the Macodes petola. Upon drying of the silk film (negative replica), this was peeled off and stored at room temperature until further use. Positive replicas were obtained by first casting a PDMS solution directly on the leaf, letting it dry, peeling it off, and by using it as a mold to cast silk solution in it. Silk films with Rhodamine 6G were fabricated by casting the silk-rhodamine solution (C_{rhodamine}=0.1 mg/ml) either in polystyrene Petri dishes or on the PDMS negative replica to give either smooth or patterned films, respectively.

Plant photographs. Digital macroscopic images of the plant specimen were taken with a smartphone (iPhone 11 Pro, Apple) and with a DSLR (digital single-lens reflex) camera (Canon Rebel EOS-Tli) adjusted for exposure and contrast. For the UV light images, a 3-wavelength UV lamp (Upland, CA 91786 USA, λ₁=365 nm, λ₂=302 nm, λ₃=254 nm) was used.

Optical microscopy and spectroscopy. Optical microscopy and micro-spectroscopy were performed on a customized Olympus upright Olympus BX51 microscope coupled with a DSLR (digital single-lens reflex) camera (Canon Rebel EOS-T1i) using a halogen lamp (U-LH100L-3) as a light source in Koehler illumination. To perform micro-spectroscopy to quantify the reflectance of the golden veins, the microscope was coupled to a spectrometer (Ocean Optics, USB2000) using an optical fiber (200 μm core size). The reflectance spectra were normalized against a silver mirror standard (Thorlabs, PF10-03-P01, avg. reflectance >97.5% for λ=450-2000 nm). Bright field reflection images were collected using a 10× (Olympus, MPlan N, NA=0.25), and 20× (Olympus, LUCPlanFL N, NA=0.45) objectives. The spectra were smoothed and plotted using the software MATLAB R2023a. To perform k-space imaging the microscope was equipped with a Bertrand Lens (U-P515, Olympus) and a 40× objective (Olympus, LUCPlan FL N, NA=0.6) was used. A 1.2 μm grating (Digital Optics Corp.) was used to calibrate the k-space.

Plant laser imaging. Wavelength-dependent imaging of the leaf was carried out using a bespoke setup. A stereo microscope (SZ-STU2, Olympus) was coupled to a DSLR (digital single-lens reflex) camera (Canon Rebel EOS-Tli) using a laser in illumination. Green light was provided by using a λ=543.5 nm, 1 mW Melles Griot laser, while red light by using a HeNe laser (λ=632.8 nm, 10 mW Melles Griot). The laser light was delivered to the leaves through an optical fiber (SMF-28-100, Thorlabs).

Multispectral imaging. To perform multispectral analysis of the Macodes petola leaves, an Olympus BX51 microscope equipped with a halogen lamp (U-LH100L-3) as a light source was coupled to a multispectral camera (CRI Nuance EX). A portion of the leaves containing the reticulated veins was cut from the main leaf using a surgical punch and was observed using a 40× objective (Olympus, LUCPlanFL N, NA=0.6) in bright field reflection. The spectral maps were acquired in the range λ=450-700 nm with a step size of 2 nm. To perform multispectral analysis of the Macodes petola plant the multispectral camera (CRI Nuance EX) was directly coupled to a Computar 55 mm objective. The spectral maps were acquired in the range λ=450-750 nm with a step size of 5 nm. The software Nuance 3.0.2 was used to acquire and unmix the maps in different spectral components and the software MATLAB R2023a was used to analyze the data.

Fluorescence imaging. To perform fluorescence analysis of the Macodes petola leaves, an Olympus IX71 microscope equipped with a mercury lamp as a light source was coupled to a multispectral camera (CRI Nuance Ex). A portion of the leaves containing the reticulated veins was cut from the main leaf using a surgical punch and was observed using a 10× objective (Olympus, UPlan FL N, NA=0.3) in bright field fluorescence. The spectral maps were acquired in the range λ=520-850 nm with a step size of 5 nm, using an excitation filter (Ex 480/40 nm) to filter the incoming light. The software Nuance 3.0.2 was used to acquire and unmix the maps in different spectral components and the software MATLAB R2023a was used to analyze the data. The fluorescence analysis of the silk films with Rhodamine 6G was carried out using the same setup with the spectral maps being acquired in the range λ=520-600 nm with a step size of 5 nm.

Scanning electron microscopy. Imaging of the silk replicas was performed by mounting the free-standing replica films on aluminum stubs using carbon tape to be either at 90° (top view imaging) or at 0° (cross-sectional imaging) with respect to the electron beam. To ensure electrical conductivity, the stubs were sputtered with ~8-10 nm of gold using an Emitech SC7620 sputter coater. The specimens were imaged using a Zeiss EVO MA10 Scanning Electron Microscope (SEM) with a secondary electron detector at 10 kV with 6 to 7.5 mm as the working distance.

Cryo-scanning electron microscopy. Cryo-scanning electron microscopy imaging of the fresh Macodes leaves was carried out as it follows. A small portion of the leaf was isolated using a surgical punch, mounted on a cryo holder and quenched in liquid nitrogen. The leaf was maintained under cryogenic conditions and cut with a blade within a Quorum Chamber before being coated with a thin layer of gold (~5 nm) before being transferred to a Zeiss Sigma SEM operating with a secondary electron at 2 kV and 4-7 mm as working distance.

Diffraction patterns. The Macodes replica were illuminated by a red laser pointer (λ=632 nm) positioned normally with respect to the patterned replica. The transmission diffraction patterns were collected on a white screen positioned normally with respect to the samples and captured using a smartphone camera (iPhone 11 Pro, Apple) located in front of the screen. The software ImageJ was used to analyze the patterns.

Laser light propagation in silk films. The silk films were illuminated by a red laser beam (λ=650 nm, Shenzhen Yize Technology Co., XS09) positioned normally with respect to the films at a distance d₁=7 cm. The transmission patterns were collected on a screen positioned at a distance d₂=11 cm away from the samples and captured using a camera (Canon Rebel EOS Tli) located at the back of the screen. The attenuation of the laser beam caused by the interaction with the silk films was measured using a power meter (Thorlabs, PM100A) equipped with a thermal power sensor head (Thorlabs, S310C) placed at a distance d₂=13 cm away from the laser. The silk films were positioned at a distance d₃=12 cm from the laser.

Optical modelling. Light interaction with the cells in the upper epidermis of the leaves was simulated using a Finite-Difference Time-domain (FDTD) method using the software FDTD Lumerical Solutions (Lumerical Inc.)⁴⁴ The cells' shape was simplified to hemispheres (r=60 μm) arranged in a hexagonal lattice; the refractive index of the cells was set to either n=1.55 (dome) or to n_{celluose}=1.55 and n_{water}=1.33 (cellulose shell and water core). As controls, a flat film (n=1.55) and a film decorated with cells with conical shapes arranged within the same lattice (n=1.55) were used. A plane wave (λ=400 to 700 nm) incident normal to the films and propagating in air (n=1) was used as light source and a video monitor positioned normal to the films was used to study the electromagnetic fields interaction with the films. Alternatively, plane waves with λ=632±50 nm and λ=543±50 nm were used to investigate the wavelength-dependent response of the cells. Boundary conditions were set to periodic and absorbing (perfectly matching layer), respectively, for the direction normal and parallel to the light source propagation.

1. A method of producing a replica of a leaf having a lattice structure including cells having a partially-spherical portion, the method comprising:
casting a silk fibroin solution directly on the leaf;
allowing the silk fibroin solution to dry at room temperature to form a silk film;
after the silk film is dry, peeling the silk film from the leaf to obtain the replica.

2. The method of claim 1, wherein the silk fibroin solution has a concentration of between 2 wt % and 15 wt %, including a concentration of C=8.48 wt %.

3. The method of claim 1, wherein the leaf is from a jewel orchid species from a genus selected from the group consisting of genus Anoectochilus, Dossinia, Goodyera, Ludisia, Macodes, Rhomboda, and Odontochilus.

4. The method of claim 3, wherein the jewel orchid is Macodes petola.

5. The method of claim 1, wherein the replica has a replica lattice structure corresponding to the lattice structure of the leaf.

6. The method of claim 5, wherein light propagation through the replica results in a far field diffraction pattern associated with a regularity of the replica lattice structure of the replica.

7. The method of claim 6, wherein the far field diffraction pattern includes a central peak surrounded by six other maxima.

8. The method of claim 5, further comprising:
tuning a parameter of the lattice structure of the replica to control light guiding and redistribution, wherein tuning is optionally performed by tailoring one or more properties or components of the silk fibroin solution or adjusting a degree of crystallinity of the replica.

9. The method of claim 5, further comprising:
tuning a cell shape of the replica lattice structure of the replica to control light guiding and redistribution.

10. The method of claim 5, further comprising:
tuning a composition of the silk fibroin solution to control light guiding and redistribution.

11. The method of claim 1, wherein the lattice structure of the replica includes a hexagonal pattern and a partially spherical morphology corresponding to the leaf.

12. The method of claim 1, wherein the replica is a negative replica of the leaf.

13. The method of claim 1, wherein the replica is a free-standing film with an integrated optical network.

14. The method of claim 1, further comprising:
obtaining the silk fibroin solution by:
sieving silk fibroin powder through a 300 μm sieve;
dissolving the sieved silk fibroin powder in double distilled water to produce an intermediate solution;
centrifuging the intermediate solution at 10,200 rpm for 20 minutes at 4° C. to produce a clear supernatant; and
collecting and filtering the clear supernatant to obtain the silk solution.

15. The method of claim 1, further comprising obtaining the silk fibroin solution by dissolving and solubilizing silk cocoons.

16. The method of claim 1, further comprising obtaining the silk fibroin solution by dissolving a silk powder in water.

17. A method of producing a positive replica of a leaf having a lattice structure including cells having a partially spherical portion, the method comprising:
casting an elastomeric material solution directly on the leaf;
allowing the elastomeric material solution to dry at room temperature to form a mold;
after the mold is dry, peeling the mold from the leaf.

18.-20. (canceled)

21. The method of claim 17, further comprising:
casting a silk fibroin solution on the mold;
allowing the silk fibroin solution to dry at room temperature to form a silk film; and
after the silk film is dry, removing the silk film from the mold to obtain the positive replica.

22. The method of claim 21, further comprising:
at least one of tuning a parameter of the replica lattice structure of the positive replicas cell shape of the replica lattice structure of the positive replica, or a composition of the silk fibroin solution to control light guiding and redistribution.

23.-33. (canceled)

34. A replica of a leaf having a lattice structure including cells having a partially spherical portion, the replica comprising:
a biopolymer matrix having a matrix lattice structure corresponding to the lattice structure of the leaf,
wherein the biopolymer matrix includes at least one optical property corresponding to an optical property of the leaf.

35.-61. (canceled)
